(19) United States
(12) Patent Application Publication
(10) Pub. No.: US 2025/0261969 A1
(43) Pub. Date: Aug. 21, 2025

(54) MAGNETICALLY ACTUATED SYSTEMS AND DEVICES FOR PERFORMING DISTRACTION HISTOGENESIS SURGICAL PROCEDURES

(71) Applicant: BioDynamik, Inc., Lake Forest, CA (US)

(72) Inventors: Shanbao Cheng, Lake Forest, CA (US); Emmon Johnny Chen, Lake Forest, CA (US)

(73) Assignee: BioDynamik, Inc., Lake Foresst, CA (US)

(21) Appl. No.: 19/054,227

(22) Filed: Feb. 14, 2025

Related U.S. Application Data
(60) Provisional application No. 63/554,970, filed on Feb. 17, 2024.

(51) Int. Cl. A61B 17/66; A61B 17/00

(52) U.S. Cl. CPC A61B 17/66; A61B 2017/00477; A61B 2017/00876

(57) ABSTRACT

Systems, methods, and devices for performing a distraction histogenesis surgical procedure. A system includes an anchor screw comprising a hollow interior defined by a sidewall, wherein the anchor screw comprises internal actuation threading attached to an interior surface of the sidewall and further comprises external anchor threading attached to an exterior surface of the sidewall. The system includes an actuation screw comprising a screw shaft that comprises external actuation threading. The system includes a magnet coupled to the actuation screw. The system is such that the external actuation threading of the screw shaft corresponds with the internal actuation threading of the anchor screw. The system is such that rotation of the magnet causes synchronous rotation of the actuation screw.

### TECHNICAL FIELD

The disclosure relates generally to systems, methods, and devices for surgical procedures, and relates specifically to systems, methods, and devices for distraction histogenesis procedures such as periosteal distraction.

### BACKGROUND

In some cases, a patient benefits from perfusion improvement and tissue regeneration to aid in recovery from vascular damage, tissue trauma, bone defects, nerve damage, and other wounds. Tissue regeneration can be a major challenge for patient care and can be particularly difficult for patients experiencing chronic wounds, ischemic diseases, diabetic foot ulcers, bone defects, and other health challenges.

Tissue regeneration is particularly difficult to trigger when treating diabetic patients, and especially when treating diabetic foot ulcers. Currently, there are few known successful treatments for recalcitrant diabetic foot ulcers. Ongoing research suggests there may be some success in topical therapies and oxygen treatments, but these treatments show limited success.

Additionally, it can be difficult to successfully trigger bone regeneration in response to a bone defect, fracture, or other trauma. Commonly used therapies for bone regeneration include bone graft substitutes, guided bone regeneration (GBR), distraction osteogenesis (DO), and periosteal distraction osteogenesis (PDO). PDO is a combination of tissue expansion and GBR and is implemented to create an artificial space between the bone surface and periosteum layer by expanding the periosteum, muscle, and skin at the same time.

Traditional devices for performing distraction histogenesis, and specifically for performing periosteal distraction osteogenesis, include numerous external components that may be bothersome and painful for the patient, can be difficult and time consuming to install, require numerous component implanted into patient bone tissue, and can be difficult to adjust as time goes on. What is needed are systems, methods, and devices for performing distraction histogenesis that improve patient outcomes, reduce surgery complexity, reduce the number of percutaneous sites, and reduce the quantity of cumbersome and painful external components.

In view of the foregoing, disclosed herein are systems, methods, and devices for improved surgical procedures, and specifically for improved distraction histogenesis procedures such as periosteal distraction.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIG. 1 is a perspective view of a system for performing a distraction histogenesis surgical procedure, and specifically illustrates wherein an actuation screw is partially screwed into an anchor screw such that the device is partially distracted;

FIG. 2 is a perspective view of a system for performing a distraction histogenesis surgical procedure, and specifically illustrates wherein an actuation screw is partially screwed into an anchor screw such that the device is partially distracted;

FIG. 3 is a straight-on side view of a system for performing a distraction histogenesis surgical procedure, and specifically illustrates wherein an actuation screw is partially screwed into an anchor screw such that the device is partially distracted;

FIG. 4 is a perspective cross-sectional view of a system for performing a distraction histogenesis surgical procedure, and specifically illustrates wherein an actuation screw is partially screwed into an anchor screw such that the device is partially distracted;

FIG. 5 is a perspective view of a system for performing a distraction histogenesis surgical procedure, wherein an actuation screw is fully screwed into an anchor screw such that the device is fully retracted and has its minimum total vertical length;

FIG. 6 is a straight-on side view of a system for performing a distraction histogenesis surgical procedure, wherein an actuation screw is fully screwed into an anchor screw such that the device is fully retracted and has its minimum total vertical length;

FIG. 7 is a perspective cross-sectional view of a system for performing a distraction histogenesis surgical procedure, wherein an actuation screw is fully screwed into an anchor screw such that the device is fully retracted and has its minimum total vertical length;

FIG. 8 is a straight-on side view of a system for performing a distraction histogenesis surgical procedure, wherein the device includes a distraction plate, and wherein an actuation screw is fully screwed into an anchor screw such that the device is fully retracted and has its minimum total vertical length;

FIG. 9 is an overhead perspective view of a system for performing a distraction histogenesis surgical procedure, wherein the device includes a distraction plate, and wherein an actuation screw is fully screwed into an anchor screw such that the device is fully retracted and has its minimum total vertical length;

FIG. 10 is an underside perspective view of a system for performing a distraction histogenesis surgical procedure, wherein the device includes a distraction plate, and wherein an actuation screw is fully screwed into an anchor screw such that the device is fully retracted and has its minimum total vertical length;

FIG. 11 is a cross-sectional perspective view of a system for performing a distraction histogenesis surgical procedure, wherein the device includes a distraction plate, and wherein an actuation screw is fully screwed into an anchor screw such that the device is fully retracted and has its minimum total vertical length;

FIG. 12 is a cross-sectional perspective view of a system for performing a distraction histogenesis surgical procedure, wherein the device includes a distraction plate, and wherein an actuation screw is fully screwed into an anchor screw such that the device is fully retracted and has its minimum total vertical length;

FIG. 13 is a schematic illustration of a cross-sectional straight-on side view of an anti-rotational assembly;

FIG. 14 is a perspective view of a system for performing a distraction histogenesis surgical procedure, wherein the device includes a magnetic core and a distraction plate, and wherein an actuation screw is fully screwed into an anchor screw such that the device is fully retracted and has its minimum total vertical length;

FIG. 15 is a straight-on side view of a system for performing a distraction histogenesis surgical procedure, wherein the device includes a magnetic core and a distraction plate, and wherein an actuation screw is fully screwed into an anchor screw such that the device is fully retracted and has its minimum total vertical length;

FIG. 16 is an aerial top-down view of a system for performing a distraction histogenesis surgical procedure, wherein the device includes a magnetic core and a distraction plate, and wherein an actuation screw is fully screwed into an anchor screw such that the device is fully retracted and has its minimum total vertical length;

FIG. 17 is a cross-sectional perspective view of a system for performing a distraction histogenesis surgical procedure, wherein the device includes a magnetic core and a distraction plate, and wherein an actuation screw is fully screwed into an anchor screw such that the device is fully retracted and has its minimum total vertical length;

FIG. 18 is a cross-sectional perspective view of a system for performing a distraction histogenesis surgical procedure, wherein the device includes a magnetic core and a distraction plate, and wherein an actuation screw is fully screwed into an anchor screw such that the device is fully retracted and has its minimum total vertical length;

FIG. 19 is a cross-sectional perspective view of a system for performing a distraction histogenesis surgical procedure, wherein the device includes a magnetic core and a distraction plate, and wherein an actuation screw is fully screwed into an anchor screw such that the device is fully retracted and has its minimum total vertical length;

FIG. 20 is a perspective view of a system for performing a distraction histogenesis surgical procedure, wherein the device includes a magnetic core, and wherein an actuation screw is fully screwed into an anchor screw such that the device is fully retracted and has its minimum total vertical length;

FIG. 21 is a perspective cross-sectional view of a system for performing a distraction histogenesis surgical procedure, wherein the device includes a magnetic core, and wherein an actuation screw is fully screwed into an anchor screw such that the device is fully retracted and has its minimum total vertical length;

FIG. 22 is a straight-on side view of a system for performing a distraction histogenesis surgical procedure, wherein the system includes an external magnetic controller (EMC) that forms a magnetic coupling with a magnet of a distraction device;

FIG. 23 is a perspective view of a system for performing a distraction histogenesis surgical procedure, wherein the system includes an EMC that forms a magnetic coupling with a magnet of a distraction device;

FIG. 24 is an overhead perspective view of an EMC configured to form a magnetic coupling with a magnet of a distraction device;

FIG. 25 is a cross-sectional aerial top-down view of an EMC configured to form a magnetic coupling with a magnet of a distraction device;

FIG. 26 is a cross-sectional perspective view of a system for performing a distraction histogenesis surgical procedure, wherein the system includes an EMC that forms a magnetic coupling with a magnet of a distraction device;

FIG. 27A is a schematic illustration of an aerial top-down view of an EMC;

FIG. 27B is a schematic illustration of a cross-sectional straight-on side view of an EMC including an external magnet;

FIG. 27C is a schematic illustration of a cross-sectional straight-on side view of an EMC including an external magnet and a motor;

FIG. 28 is a schematic illustration of a cross-sectional straight-on side view of an EMC including electromagnets;

FIGS. 29A-29F are schematic illustrations of a system for driving an implant magnet of a distraction device with an EMC comprising electromagnets and a Hall effect sensor or Hall effect array;

FIG. 30 is a schematic illustration of a straight-on cross-sectional side view of an EMC including an electromagnet;

FIG. 31A is a schematic illustration of a cross-sectional aerial top-down view of a magnet of a distraction device or an external magnet of an EMC that includes two poles;

FIG. 31B is a schematic illustration of a cross-sectional aerial top-down view of a magnet of a distraction device or an external magnet of an EMC that includes four poles;

FIG. 31C is a schematic illustration of a cross-sectional aerial top-down view of a magnet of a distraction device or an external magnet of an EMC that includes eight poles;

FIG. 32A is a schematic illustration illustrating magnetic attraction between a magnet of a distraction device and an external magnet of an EMC;

FIG. 32B is a schematic illustration illustrating magnetic attraction between a magnet of a distraction device and an external magnet of an EMC, and further illustrates synchronous rotation of the magnet of the distraction device in response to clockwise rotation of the external magnet of an EMC;

FIG. 32C is a schematic illustration illustrating magnetic attraction between a magnet of a distraction device and an external magnet of an EMC, and further illustrates synchronous rotation of the magnet of the distraction device in response to counterclockwise rotation of the external magnet of an EMC;

FIG. 33 is a schematic illustration of a system for performing a distraction histogenesis surgical procedure on patient tissue;

FIGS. 34A and 34B are schematic illustrations of a system and process for performing a distraction histogenesis surgical procedure with an EMC;

FIGS. 35A and 35B are schematic illustrations of a system and process for performing a distraction histogenesis surgical procedure with an EMC;

FIGS. 36A and 36B are schematic illustrations of a system and process for performing a distraction histogenesis and osteotomy surgical procedure with an EMC;

FIGS. 37A-37C are schematic flow chart diagrams of a method for performing a distraction histogenesis surgical procedure;

FIGS. 38A-38E are schematic flow chart diagrams of a method for performing a distraction histogenesis surgical procedure;

FIG. 39 is a schematic block diagram of a system for monitoring health data and remotely actuating adjustment of a distraction device; and

FIG. 40 is a schematic block diagram of an example computing device.

### DETAILED DESCRIPTION

Described herein are systems, methods, and devices for promoting revisualization, improving perfusion and triggering tissue regeneration. The disclosures herein may be implemented to trigger regeneration of bone tissue, vascular tissue, microvascular tissue, skin tissue, nerve tissue, and other tissue types. The systems, methods, and devices described herein may be utilized to perform distraction histogenesis surgical procedures on various tissue types and may specifically be utilized to perform periosteal distraction osteogenesis to trigger regeneration of vascular and soft tissues.

What is needed are improved systems, methods, and devices for periosteal distraction that reduce the risks of infections, reduce the risks of device complications, reduce surgery complexity, reduce the quantity of percutaneous sites prone to infection, reduce the number of external components that may be cumbersome and painful for the patient, and reduce the complexity of tasks performed after surgery. Additionally, what is needed are systems, methods, and devices for triggering regeneration of non-bone tissue types through distraction histogenesis, including, for example, promoting revascularization for the healing of diabetic foot ulcers and other ischemic limb diseases.

In view of the foregoing, described herein systems, methods, and devices for initiating tissue growth through distraction histogenesis. The distraction histogenesis techniques described herein may be utilized to treat tissue defects by raising systemic growth factor levels. One or more of the devices described herein can be coupled to many different tissues in the body, because the devices are internal and implantable.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Before the structures, systems, methods, and compositions described herein are disclosed, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials, or steps and those that do not materially affect the basic and novel characteristic or characteristics of the claimed disclosure.

Unless defined otherwise, all technical and scientific terms used herein have the same meanings as commonly understood by one of ordinary skill in the art to which this disclosure pertains and belongs.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

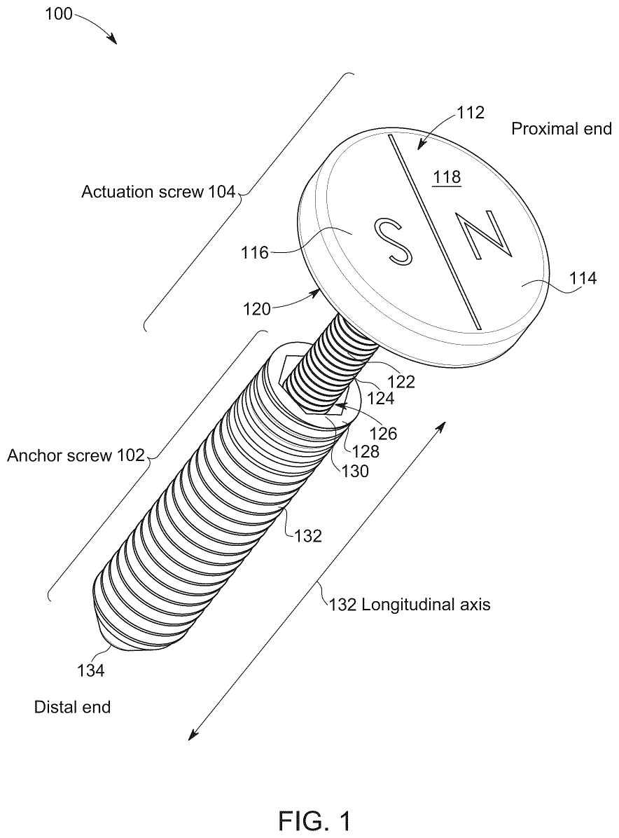

Referring now to the figures, FIG. 1 is a perspective view of a system 100 for performing a distraction histogenesis surgical procedure. The system 100 may be utilized to perform various distraction procedures, including those performed on soft tissues. The system 100 may specifically be utilized to perform a periosteal distraction surgical procedure to lift a periosteum tissue away from adjacent bone tissue and thereby trigger tissue generation.

The system 100 is configured to be implanted into a patient during a surgical procedure. After implantation, the system 100 may be distracted or retracted according to the patient's needs. In an example use-case, the system 100 is implanted in between bone tissue and a periosteum layer of a patient. The surgical site may be fully closed over the system 100 to prevent infection, and this may include suturing shut one or more of the periosteum or skin layers over the system 100. The system 100 may then be magnetically actuated without physically touching the device. The magnetic coupling may be performed with an external magnetic controller (EMC) as described herein, or another similar device. A user may thereby utilize an external magnet or electromagnet to distract or retract the system 100 in a controlled manner while the system 100 is installed in the patient.

The system 100 includes two primary components, including an anchor screw 102 and an actuation screw 104. The system 100 includes a longitudinal axis, and the actuation screw 104 is configured to move up and down along the longitudinal axis and relative to the anchor screw 102. When the actuation screw 104 is distracted from the anchor screw 102 as shown in FIG. 1, the system 100 has a longer total length. When the actuation screw 104 is fully retracted into the anchor screw 102, the system 100 has its minimum total length.

As shown in FIG. 1, at least a portion of the actuation screw 104 is configured to be disposed within and encased by the anchor screw 102. The anchor screw 102 is configured to be screwed into a first tissue of a patient and may specifically be configured to be screwed into bone tissue. The actuation screw 104 is configured to move up and down relative to a longitudinal axis of the anchor screw 102 to adjust an overall length of the system 100. The vertical position of the actuation screw 104 relative to the anchor screw 102 may be adjusted over time to cause distraction or retraction of a second tissue of the patient.

The actuation screw 104 includes a screw shaft 122 and a plate magnet 112 attached to the screw shaft 122. The plate magnet 112 includes at least a first pole 114 and a second pole 116, and the plate magnet 112 may include additional poles depending on the implementation. The plate magnet 112 may have, for example, four poles, six poles, eight poles, and so forth, without departing from the scope of the disclosure. In some cases, it may be desirable to design the plate magnet 112 with a higher quantity of poles to enable more granularity in unscrewing and screwing the actuation screw 104.

The first pole 114 and the second pole 116 comprise opposite magnetic polarity relative to one another. The plate magnet 112 includes a magnet top surface 118 and a magnet bottom surface 120 (not visible in the view illustrated in FIG. 1) that is opposite to the magnet top surface 118. As shown in FIG. 1, the first pole 114 and the second pole 116 are located adjacent to one another such that each of the first pole 114 and the second pole 116 is located at least on the magnet top surface 118 of the plate magnet 112. The screw shaft 122 is attached to the magnet bottom surface 120 of the plate magnet 112. The screw shaft 122 comprises external threading 124 that is configured to engage with internal threading disposed within a hollow interior of the anchor screw 102.

The anchor screw 102 comprises the longitudinal axis running along a length of the anchor screw 102. The actuation screw 104 is configured to engage with the anchor screw to adjust a vertical position of the plate magnet 112 along the longitudinal axis of the anchor screw 102. The anchor screw 102 includes a hollow interior 126 defined by a sidewall 128. The anchor screw 102 includes internal threading (not visible in FIG. 1) on an interior surface of the sidewall 128. The internal threading of the anchor screw 102 corresponds with the external threading 124 of the screw shaft 122 of the actuation screw 104. The anchor screw 102 includes external threading 132 on an exterior surface of the sidewall 128. The external threading 132 is configured to engage with a first tissue of a patient to anchor the anchor screw 102 to the first tissue. The anchor screw 102 include a tip 134 disposed at a proximal (i.e., the end proximal to an interior of a patient when the anchor screw 102 is installed into the first tissue of the patient).

The anchor screw 102 includes a socket interface 130 disposed at a proximal end of the anchor screw 102, and the socket interface 130 provides a means to directly rotate the anchor screw 102. The socket interface 130 may include a recess comprising a polygonal cross-sectional geometry such as a quadrilateral, pentagonal, hexagonal, and so forth, cross-sectional geometry. The polygonal cross-sectional geometry of the socket interface 130 enables a machine or human user to get sufficient torque when directly screwing the anchor screw 102.

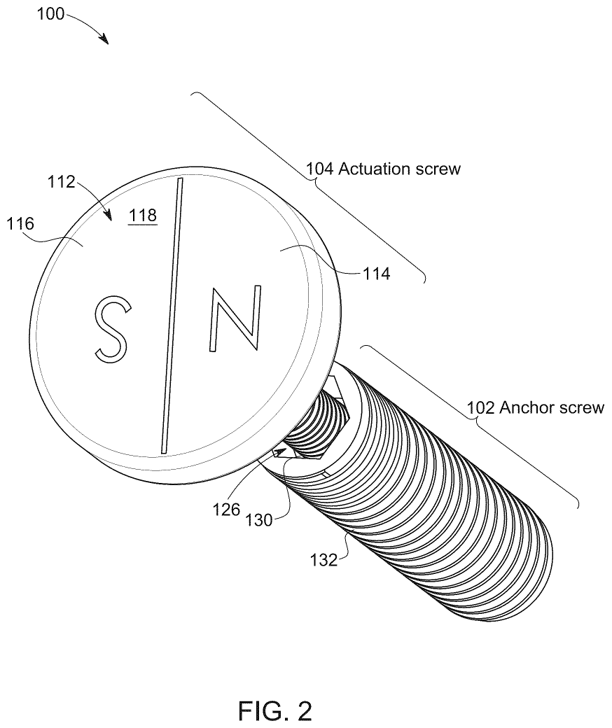

FIG. 2 is a perspective view of the system 100 for performing a distraction histogenesis surgical procedure. The plate magnet 112 of the actuation screw 104 may comprise a strong magnet that extends the entire thickness of the magnet plate. The strength of the plate magnet 112 may be sufficient to ensure the plate magnet 112 maintains stable magnetic coupling with an external magnet that is located external to the patient tissue, even when the magnet is installed within the patient's body. In an implementation, the plate magnet 112 is installed underneath several layers of patient tissue, including, for example, periosteum, fat, tendons, ligaments, and skin. The plate magnet 112 may be sufficiently strong to maintain a magnetic coupling with a separate external magnet that is located entirely external to the patient's body.

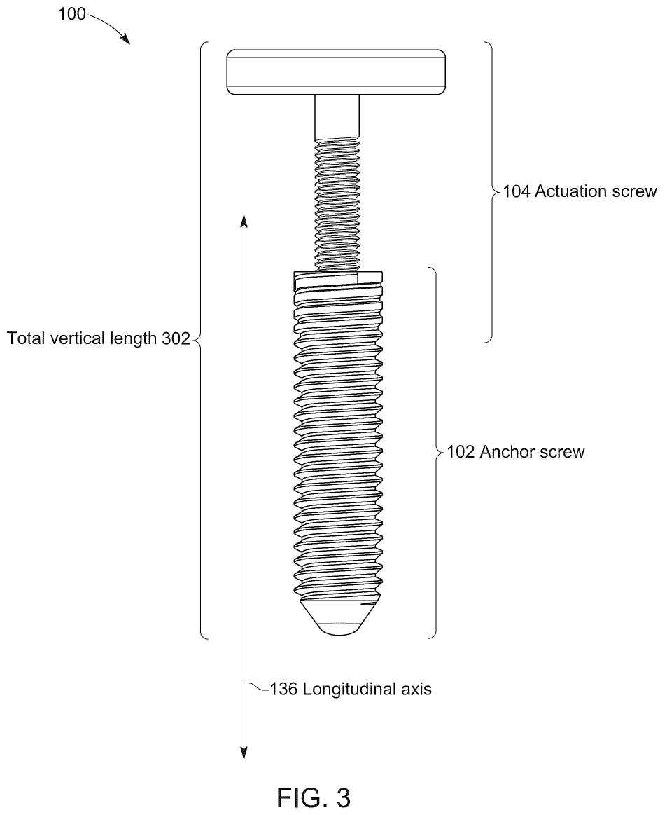

FIG. 3 is a straight-on side view of the system 100 for performing a distraction histogenesis surgical procedure. Like FIGS. 1 and 2, FIG. 3 also illustrates wherein the actuation screw 104 is partially distracted or screwed out of the anchor screw 102. The position of the actuation screw 104 along the longitudinal axis of the anchor screw 102 adjusts the total vertical length 302 of the system 100.

In practice, the anchor screw 102 may be fully screwed into a first tissue of a patient, such as a bone tissue, such that only the distal end of the anchor screw is visible or located outside the first tissue. The actuation screw 104 may then be fully screwed into the anchor screw 102 to achieve a minimum total vertical length 302 for the system 100. The actuation screw 104 may be gradually unscrewed from the anchor screw 102 to increase the total vertical length 302 of the device, and thereby cause distraction of a second tissue that is disposed above the actuation screw 104. The actuation screw 104 may be screwed further into the anchor screw 102 at any time to cause retraction of the second tissue.

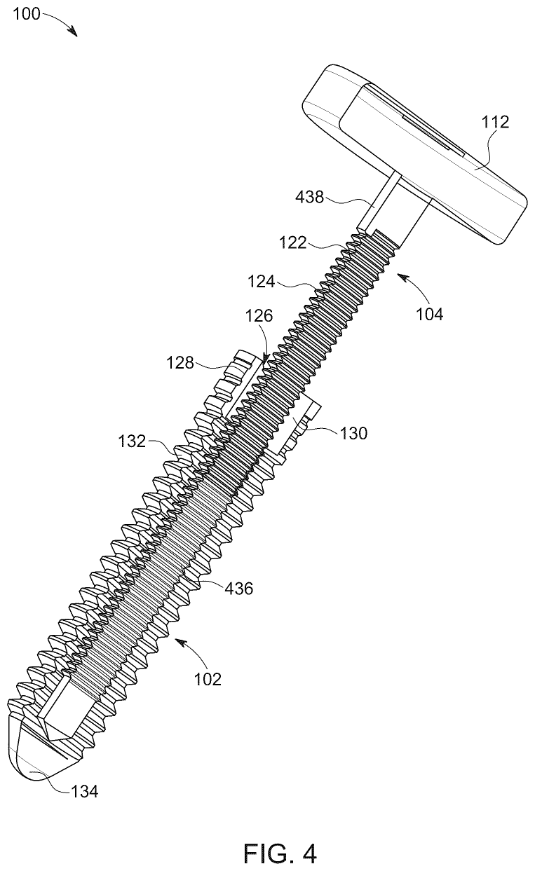

FIG. 4 is a perspective cross-sectional view of the system 100 for performing a distraction histogenesis surgical procedure, wherein the actuation screw 104 is partially distracted from the anchor screw 102. The cross-sectional view in FIG. 4 enables viewing of internal threading 436 disposed along an interior side of the sidewall 128 of the anchor screw 102.

The actuation screw 104 is coupled to the anchor screw 102 by interfacing the external threading 124 of the actuation screw 104 with the internal threading 436 of the anchor screw 102. Because the actuation screw 104 is screwed into the anchor screw 102, the actuation screw 104 is prevented from sliding out of the anchor screw 102 when the plate magnet 112 of the actuation screw 104 coupled to another external magnetic component.

As shown in FIG. 4, the screw shaft 122 of the actuation screw 104 may include a smooth portion 438 disposed in between the plate magnet 112 and the portion comprising external threading 124. The actuation screw 104 may include a singular indivisible piece including each of the portion comprising external threading 124, the smooth portion 438, and the plate magnet 112.

FIG. 5 is a perspective view of the system 100 for performing a distraction histogenesis surgical procedure, wherein the actuation screw 104 is fully screwed into the anchor screw 102 such that the system 100 is fully retracted and has its minimum total vertical length.

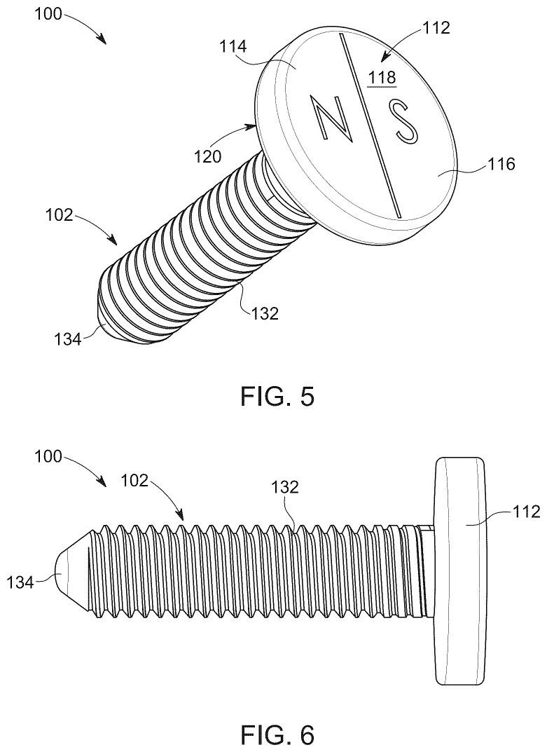

FIG. 6 is a straight-on side view of the system 100 for performing a distraction histogenesis surgical procedure, wherein the actuation screw 104 is fully screwed into the anchor screw 102 such that the system 100 is fully retracted and has its minimum total vertical length.

As shown in FIGS. 5 and 6, the screw shaft (see 122) of the actuation screw 104 is not visible when the actuation screw 104 is fully retracted into the anchor screw 102. The system 100 may include the configuration illustrated in FIGS. 5 and 6 when the system 100 is first installed into patient tissue. Depending upon the implementation and the specific health considerations of the patient, a surgeon may fully close one or more layers of patient tissue over the plate magnet 112 of the actuation screw 104. This may reduce the likelihood of infection and improve patient outcomes.

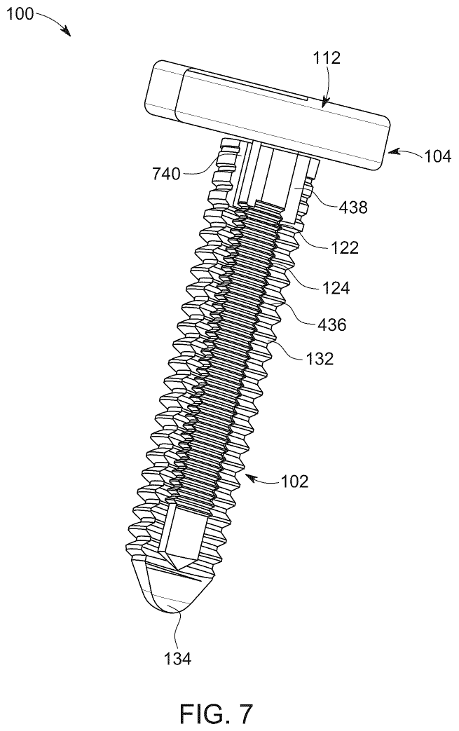

FIG. 7 is a perspective cross-sectional view of the system 100 for performing a distraction histogenesis surgical procedure, wherein the actuation screw 104 is fully screwed into the anchor screw 102 such that the system 100 is fully retracted and has its minimum total vertical length.

As shown in FIG. 7, the length of the screw shaft 122 of the actuation screw 104 may be screwed into the anchor screw 102 for most of a length of the anchor screw 102. The actuation screw 104 may be securely distracted out of the anchor screw 102 as long as the actuation screw 104 is still coupled to the anchor screw 102 through the corresponding threading. The actuation screw 104 may further be fully removed from the anchor screw 102 by unscrewing the screw shaft 122 from the anchor screw 102.

The cross-sectional view of FIG. 7 enables partial viewing of a socket head 740 of the anchor screw 102. The socket head 740 includes a polygonal cross-sectional geometry to enable a machine or human user to achieve sufficient torque to screw the anchor screw 102 into an anchoring substrate, such as bone tissue. The socket head 740 may include a quadrilateral, pentagonal, hexagonal, and so forth, polygonal cross-sectional geometry. The socket head 740 may be utilized with a corresponding driver that comprises the same polygonal cross-sectional geometry. The socket head 740 may be utilized to screw the anchor screw 102 into a substrate, or two unscrew the anchor screw 102 from the substrate, when the actuation screw 104 is not disposed within the anchor screw 102.

In an exemplary use-case, the system 100 is installed in a bone tissue of a patient to perform a periosteal distraction surgical procedure. A surgeon may first drill a pilot hole into the bone tissue, wherein the pilot is sized based on the outer diameter of the anchor screw 102. The surgeon may then screw the anchor screw 102 into the pilot hole by utilizing a driver that corresponds with the socket head 740 of the anchor screw 102. After the anchor screw 102 is installed in the bone tissue, the surgeon may then screw the actuation screw 102 into the anchor screw 104.

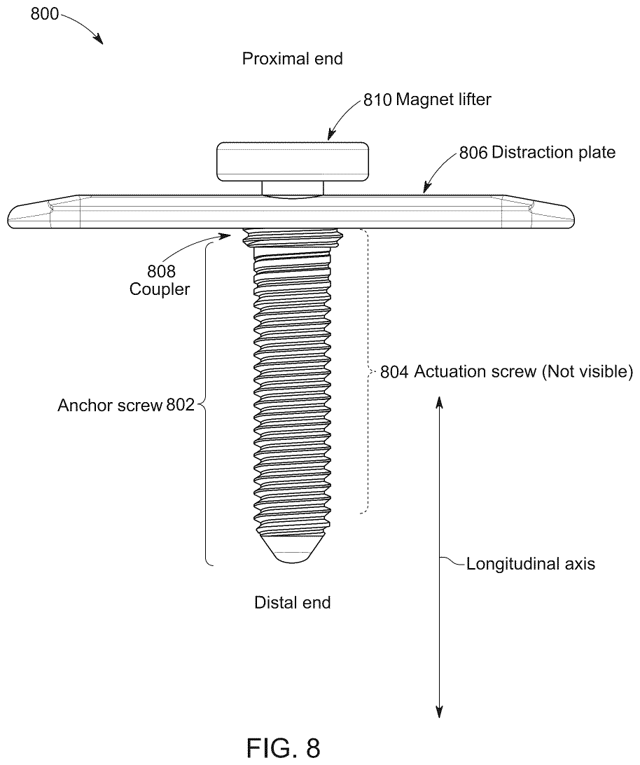

FIG. 8 is a straight-on side view of a system 800 for performing a distraction histogenesis surgical procedure. The system 800 is like the system 100 first described in connection with FIG. 1, but for the system 800 additionally including a distraction plate 806. The distraction plate 806 may aid in a distraction histogenesis procedure to lift a second tissue away from a first tissue and/or to stretch the second tissue.

Like the system 100 first described in connection with FIG. 1, the system 800 is also configured to magnetically coupled to an external magnet or electromagnet. The system 800 may be installed within a patient, and then distracted (i.e., expanded) or retracted (i.e., contracted) by establishing a magnetic coupling between the system 800 and an external magnetic controller. Thus, the system 800 may be installed within the patient and the patient's skin barrier may be fully closed over the system 800. The system 800 may still be distracted and retracted by establishing a magnetic coupling between the system 800 and the external magnetic controller that is located external to the patient's skin barrier.

The system 800 includes the anchor screw 802 that is configured to be anchored into a first tissue of a patient. The system 800 includes the actuation screw 804 that is disposed within a hollow interior formed by a sidewall of the anchor screw 802. The anchor screw 802 is similar to the anchor screw 102 first described in connection with FIG. 1 and may include all of the components associated with the anchor screw 102. The actuation screw 804 is similar to the actuation screw 104 first described in connection with FIG. 1 and may include all of the component associated with the actuation screw 104.

The system 800 may include a combined actuation screw 804 and magnet lifter 810 such that the magnet lifter 810 cannot be readily separated from the actuation screw 804. In this case, the actuation screw 804 is similar to the actuation screw 104 first described in connection with FIG. 1, wherein the actuation screw 804 is a single component including each of a screw shaft and a plate magnet.

The system 800 may include a separate actuation screw 804 and magnet lifter 810 such that the actuation screw 804 and the magnet lifter 810 constitute separate components that may interface with one another. In this case, the actuation screw 804 may include a screw shaft and a first rotational driver interface, and the magnet lifter 810 may include a plate magnet and a corresponding second rotational driver interface. In an example implementation, the actuation screw 804 may include a driver head, and the magnet lifter 810 may include a corresponding socket interface, such that the socket interface of the magnet lifter 810 may be utilized to drive rotation of the actuation screw 804.

The system 800 further includes a distraction plate 806 coupled to the actuation screw 804. The system 800 includes a coupler 808 that couples the distraction plate 806 to the actuation screw 804 in a manner that prevents the distraction plate 806 from synchronously rotating with the actuation screw 804, while ensuring the distraction plate 806 is still axially coupled to the actuation screw 804 (i.e., the distraction plate 806 and the actuation screw 804 each distract away from the anchor screw 802 and retract toward the anchor screw 802 when the actuation crew 804 is rotated).

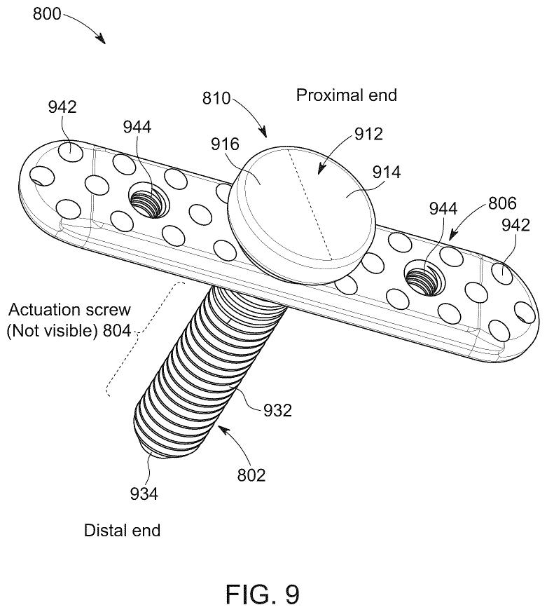

FIG. 9 is an overhead perspective view of the system 800 for performing a distraction histogenesis surgical procedure. The overhead perspective view shown in FIG. 9 enables further viewing of the distraction plate 806.

The distraction plate 806 may include a central threaded hole, which is not visible in FIG. 9 because the coupler 808 and anchor screw 802 are disposed within the central threaded hole. The central threaded hole may include internal threading that interfaces with one or more of the coupler 808 or the anchor screw 802. The distraction plate 806 may additionally include one or more peripheral threaded holes 944 comprising internal threading. The peripheral threaded holes 944 may be utilized to anchor the distraction plate 806 to a patient tissue. In an exemplary use-case, the peripheral threaded holes 944 are utilized to anchor the distraction plate 806 to osteotomized bone tissue, such that distraction of the anchor plate 806 causes likewise distraction of the osteotomized bone tissue.

The distraction plate 806 may additionally include one or more non-threaded holes 942. The non-threaded holes 942 may reduce the overall mass of the distraction plate 806 and may additionally permit patient fluids and tissues to permeate through the distraction plate 806.

The magnet lifter 810 includes a plate magnet 912 that includes at least a first pole 914 and a second pole 916. The first pole 914 and the second pole 916 comprise opposite polarity relative to one another. The plate magnet 912 may include additional poles, including, for example, four poles, six poles, eight poles, and so forth, without departing from the scope of the disclosure. In some cases, it may be desirable to design the plate magnet 912 with numerous poles to enable finer granularity when unscrewing and screwing the actuation screw.

As discussed herein, the magnet lifter 810 may comprise a separate component from the actuation screw 804, or the magnet lifter 810 and actuation screw 804 may comprise a single indivisible component. If the magnet lifter 810 and the actuation screw 804 comprise a single indivisible component, then the plate magnet 912 may be attached directly to a screw shaft of the actuation screw.

Like the anchor screw 102 first described in connection with FIG. 1, the anchor screw 802 includes external threading 932 that is configured to interface with a substrate, which may include a substrate tissue of a patient. In an exemplary use-case, the external threading 932 is utilized to install the anchor screw 802 into bone tissue of the patient. The anchor screw 804 additionally includes an anchor tip 934 disposed at a distal end of the anchor screw 804. The size and geometry of the anchor tip 934 may be optimized for the intended use-case and may specifically depend upon the identity of the substrate tissue.

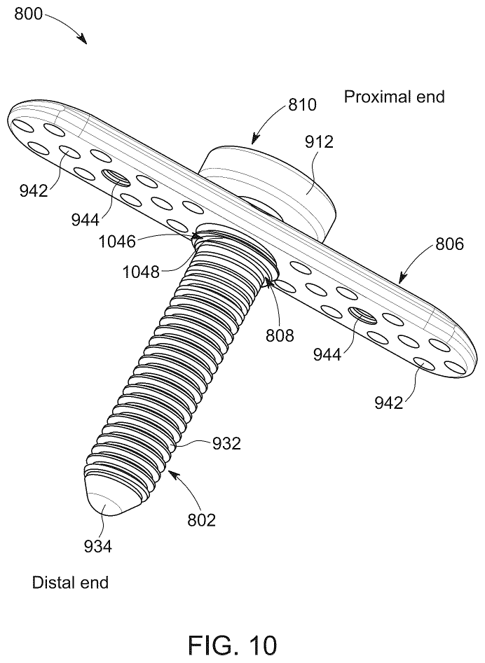

FIG. 10 is an underside perspective view of the system 800 for performing a distraction histogenesis surgical procedure. The underside view in FIG. 10 enables viewing of portions of the coupler 808.

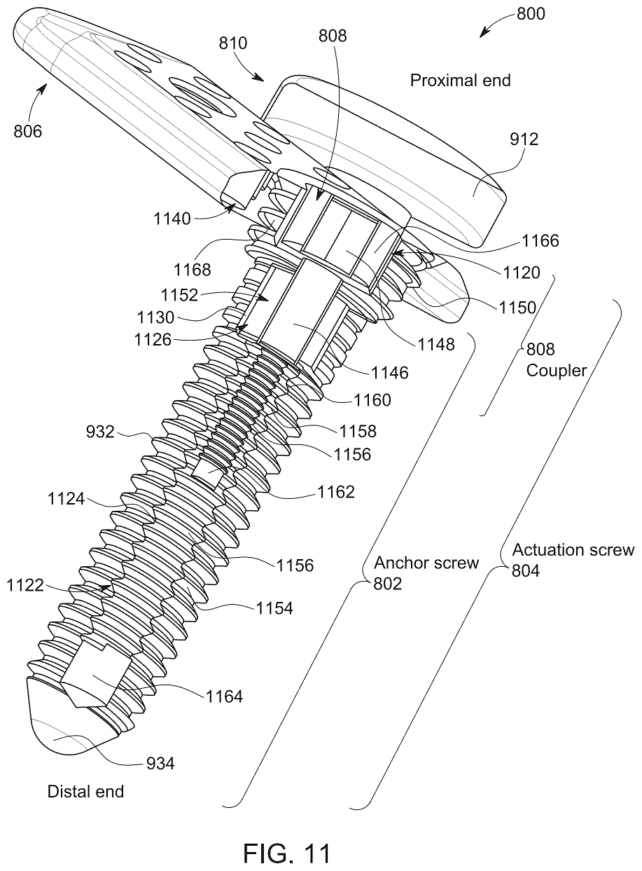

FIG. 11 is a cross-sectional perspective view of the system 800 for performing a distraction histogenesis surgical procedure. The cross-sectional view of FIG. 11 enables viewing of the actuation screw 804 for the first time, and further enables viewing of various components of the coupler 808, the magnet lifter 810, and the anchor screw 802.

The cross-sectional view of FIG. 11 enables viewing of the full length of each of the anchor screw 802, the actuation screw 804, and the coupler 808. As shown in FIG. 11, the anchor tip 934 of the anchor screw 802 serves as the distal-most point of the system 800. The actuation screw 804 is disposed within the anchor screw 802 and is further disposed through the coupler 808. The coupler 808 extends through the distraction plate 806 and partially down into the anchor screw 802 when the device is in a fully retracted position as shown in FIG. 11.

The cross-sectional view of FIG. 11 further enables viewing of various components of the actuation screw 804. The actuation screw 804 may include each of an outer screw shaft 1122 and an inner screw shaft 1160, wherein the inner screw shaft 1160 is configured to be disposed at least partially within the outer screw shaft 1122.

The system 800 includes the coupler 808 that is configured to axially couple the distraction plate 806 to the actuation screw 804 while ensuring the distraction plate 806 is not also rotationally coupled to the actuation screw 804. The coupler 808 includes an anchoring interface 1120 and a rotational interface 1126. The rotational interface 1126 is attached to the anchoring interface 1120, and the rotational interface 1126 is located distal relative to the anchoring interface 1120. The anchoring interface 1120 of the coupler 808 includes external threading 1168 that corresponds with internal threading 1150 of a central threaded hole 1140 of the distraction plate 806. The external threading 1168 of the anchoring interface 1120 thus enables the coupler 808 to be screwed into and secured to the central threaded hole 1140 of the distraction plate 806.

When the actuation screw 804 is installed within the coupler 808 and the anchor screw 802, a driver head 1148 of the actuation screw 804 may be approximately disposed within a hollow interior space defined by the anchoring interface 1120 of the coupler 808.

The rotational interface 1126 may include a polygonal cross-sectional geometry that matches the polygonal cross-sectional geometry of the socket interface 1130 of the anchor screw 802. In the example illustrated in FIG. 11, the rotational interface 1126 includes a hexagonal cross-sectional geometry, and may thus be referred to as a hex driver. The exemplary socket interface 1130 illustrated in FIG. 11 corresponds with the rotational interface 1126 and thus includes a hex socket. It should be appreciated that the rotational interface 1126 and the socket interface 1130 may include various numbers of sides as deemed appropriate, including, for example, a quadrilateral, pentagonal, hexagonal, octagonal, and so forth, polygonal cross-sectional geometry.

The coupler 808 comprises a hollow interior and a through hole, such that the coupler 808 is configured to receive the actuation screw 804. In the orientation illustrated in FIG. 11, the actuation screw 804 extends up through the coupler 808 and then interfaces with the magnet lifter 810. The coupler 808 comprises a smooth hollow interior that enables the actuation screw 804 to freely rotate within the coupler 808. The magnet lifter 810 may drive rotation of the actuation screw 804 by interfacing the driver head 1148 of the actuation screw 804. When this occurs, the actuation screw 804 may rotate freely within the coupler 808 while screwing into or out of the anchor screw 802. Thus, the coupler 808 enables the actuation screw 804 to rotate out of and into the anchor screw 802 without causing synchronous rotation of the distraction plate 806.

The coupler 808 enables the distraction plate 806 to be axially coupled to the actuation screw 804 such that distraction plate 806 moves vertically along the longitudinal axis of the system 800 while remaining rotationally stationary when the actuation screw 804 is screwed into and out of the anchor screw 802.

The outer screw shaft 1122 of the actuation screw 804 interfaces directly with the anchor screw 802. The outer screw shaft 1122 includes a sidewall that defines a hollow interior, wherein external threading 1124 is attached to an exterior surface of the outer screw shaft 1122 sidewall, and wherein internal threading 1156 is attached to an interior surface of the outer screw shaft 1122 sidewall. The proximal end of the outer screw shaft 1122 is located at a distal end of the coupler 808. The distal end of the outer screw shaft 1122 is formed by an outer actuation point 1164, which is disposed within the hollow interior of the anchor screw 802 and may nearly reach the distal-most end of the anchor screw 802. The entirety of the outer screw shaft 1122 is located distal relative to the coupler 808 and the distraction plate 806.

The external threading 1124 of the outer screw shaft 1122 is configured to interface with internal threading 1154 attached to an interior surface of a sidewall of the anchor screw 802. Thus, the outer screw shaft 1122 is configured to mate the actuation screw 804 to the anchor screw 802. The inner screw shaft 1160 does not mate directly with the anchor screw 802 and contributes to axially coupling the actuation screw 804 to the distraction plate 806 without also rotationally coupling the actuation screw 804 to the distraction plate 806.

The inner screw shaft 1160 is configured to be disposed within at least a portion of the outer screw shaft 1122. The proximal end of the inner screw shaft 1160 comprises a screw head 1148 that may be located within a portion of the coupler 808 and/or the distraction plate 806. The distal end of the inner screw shaft 1160 comprises an inner actuation point 1162 that is disposed within the hollow interior formed by the outer screw shaft 1122. The inner screw shaft 1160 is configured to be disposed through the hollow interior 1152 through-hole of the coupler 808, and thus, in combination with the coupler 808, the inner screw shaft 1160 enables the actuation screw 804 to be axially coupled to the distraction plate 806 without also being rotationally coupled to the distraction plate 806.

The inner screw shaft 1160 includes a threaded portion that includes the external threading 1158. The inner screw shaft 1160 may additionally include a smooth portion 1146. The smooth portion 1146 is configured to be disposed within the hollow interior 1152 defined by the rotational interface 1126 of the coupler 808. The smooth portion 1146 of the inner screw shaft 1160 may rotate freely within the rotational interface 1126 of the coupler 808.

The screw head 1148 of the inner screw shaft 1160 of the actuation screw 804 is configured to interface with an actuation screw coupling head 1166 of the magnet lifter 810. The actuation screw coupling head 1166 may be disposed within the hollow interior space located in between an inner surface of the anchoring interface 1120 of the coupler 808, and the outer surface of the screw head 1148 of the actuation screw 804. When the screw coupling head 1166 is located within that hollow interior space, the screw coupling head 1166 may form an interference fit with the screw head 1148 and then drive rotation of the inner screw shaft 1160 of the actuation screw 804. This may likewise drive rotation of the actuation screw 804 without driving synchronous rotation of the coupler 808 or the distraction plate 806.

The rotational interface 1126 of the coupler 808 includes a polygonal cross-sectional exterior geometry that enables the rotational interface 1126 to form a torque coupling with the corresponding polygonal cross-sectional interior geometry of a socket interface 1130 of the anchor screw 802. This enables a user to simultaneously implant the actuation screw 804 and the anchor screw 802 into a patient as a single unit.

Further as shown in the cross-sectional view, the central threaded hole 1140 of the distraction plate 806 includes internal threading 1150 that corresponds with external threading 1168 of the anchoring interface 1120 of the coupler 808. This enables the coupler 808 to be securely screwed into the distraction plate 806. Further as shown in the cross-sectional view, the anchor screw 802 includes internal threading 1154 that corresponds with the external threading 1124 of the outer screw shaft 1122 of the actuation screw 804.

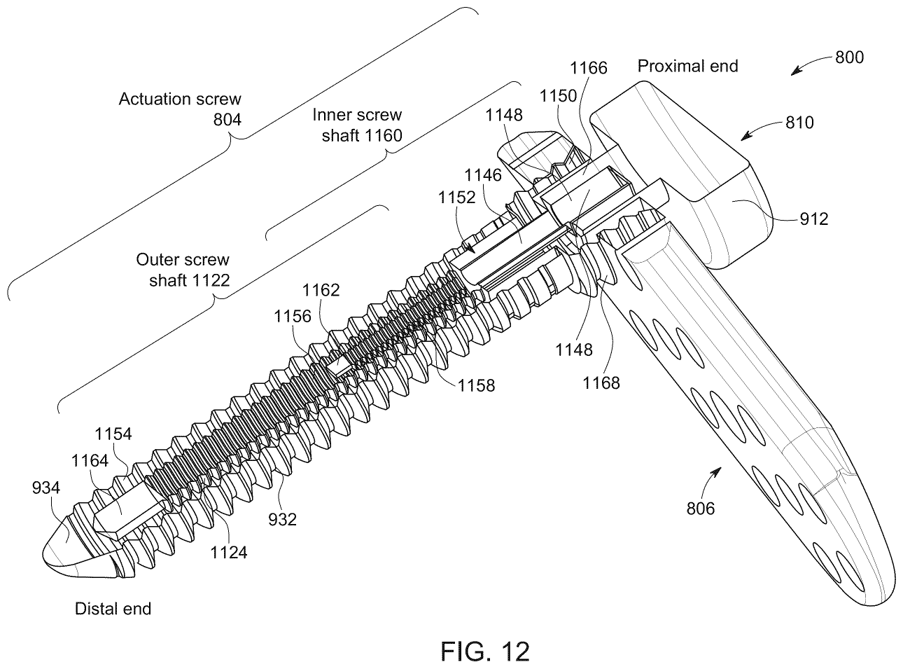

FIG. 12 is a perspective cross-sectional view of the system 800 for performing a distraction histogenesis procedure, wherein the actuation screw 804 is fully retracted into the anchor screw 802. The cross-sectional view illustrated in FIG. 12 shows that the screw head 1148 of the actuation screw 804 may have an external cross-sectional polygonal geometry that corresponds with an internal cross-sectional polygonal geometry of the screw coupling head 1166 of the magnet lifter 810. This ensures the magnet lifter 810 may have sufficient torque to rotate the actuator screw 804.

The brackets in FIG. 12 further illustrate how the combination of the outer screw shaft 1122 and the inner screw shaft 1160 make up the actuation screw 804. As shown, the inner screw shaft 1160 is partially disposed within the outer screw shaft 122, and additionally passes through the coupler 808 and terminates within the central threaded hole 1140 of the distraction plate 806.

FIG. 13 is a schematic illustration of an exploded straight-on side view of an anti-rotation assembly 1300, which is a component of the system 800 described herein. The anti-rotation assembly 1300 includes components of the actuation screw 804 (including the inner screw shaft 1160 and the outer screw shaft 1122), the coupler 808, and the distraction plate 806. The anti-rotation assembly 1300 enables the distraction plate 806 to be axially coupled to the actuation screw 804 without also being rotationally coupled to the actuation screw 804.

The anti-rotation assembly 1300 includes the actuation screw 804, which includes the inner screw shaft 1160 and the outer screw shaft 1122. The outer screw shaft 1122 comprise a diameter that is greater than a diameter of an entrance through-hole to the rotational interface 1126 of the coupler 808, and this prevents the outer screw shaft 1122 from sliding up into the coupler 808 or the distraction plate 806. The inner screw shaft 1160 comprises a diameter that is smaller than the diameter of the central threaded hole 1140 of the distraction plate 806. The diameter of the inner screw shaft 1160 is also smaller than the diameter of a hollow interior 1152 defining a through-hole extending through the entirety of the coupler 808, including the anchoring interface 1120 and the rotational interface 1126.

During assembly or installation, the inner screw shaft 1160 is slid through the through-hole defined by the sidewall of the coupler 808. The inner screw shaft 1160 is then securely coupled to the outer screw shaft 1122 by screwing the external threading 1158 of the inner screw shaft 1160 into the internal threading 1156 disposed within the hollow interior of the outer screw shaft 1122. The inner screw shaft 1160 will be fully installed within the outer screw shaft 1122 when the screw head 1148 is located approximately within the central threaded hole 1140 of the distraction plate 806 and/or when the smooth portion 1146 of the inner screw shaft 1160 is aligned with the rotational interface 1126 of the coupler 808. The outer screw shaft 1122 may include stopper that prevents the inner screw shaft 1160 from being screwed in any deeper than desired. The smooth portion 1146 of the inner screw shaft 1160 may include a diameter that is smaller than a diameter of the rotational interface 1126 of the coupler 808.

The external threading 1158 of the inner screw shaft 1160 may be tightly engaged with the internal threading 1156 of the outer screw shaft 1122 to prevent these two components of the actuation screw (see 804) from becoming separated. In some cases, the inner screw shaft 1160 may be glued into the outer screw shaft 1122 to ensure a tight and semi-permanent installation. This may help ensure that a loosening rotation of the actuation screw (see 804) to remove the actuation screw from the anchor screw (see 802) does not also loosen the inner screw shaft 1160 from the outer screw shaft 1122.

The anchoring interface 1120 of the coupler 808 includes the external threading 1168 that is configured to interface with corresponding internal threading 1150 of the central threaded hole 1140 of the distraction plate 806. This ensures the coupler 808 is releasably locked into the distraction plate 806. When the coupler 808 is further coupled to the actuation screw 804, this further ensures that the distraction plate 806 moves up and down along the longitudinal axis of the system 800 synchronously with the actuation screw 804.

The coupler 808 enables the distraction plate 806 to move vertically along the longitudinal axis of the system 800 while remaining rotationally stationary when the actuation screw 804 is screwed into and out of the anchor screw 802. In practice, if the system 800 is installed in between bone tissue and periosteum tissue to perform periosteal distraction, this ensures the distraction plate 806 remains in place underneath the periosteum tissue, and only moves outward and inward to lift and lower the patient periosteum, without rotating underneath patient periosteum.

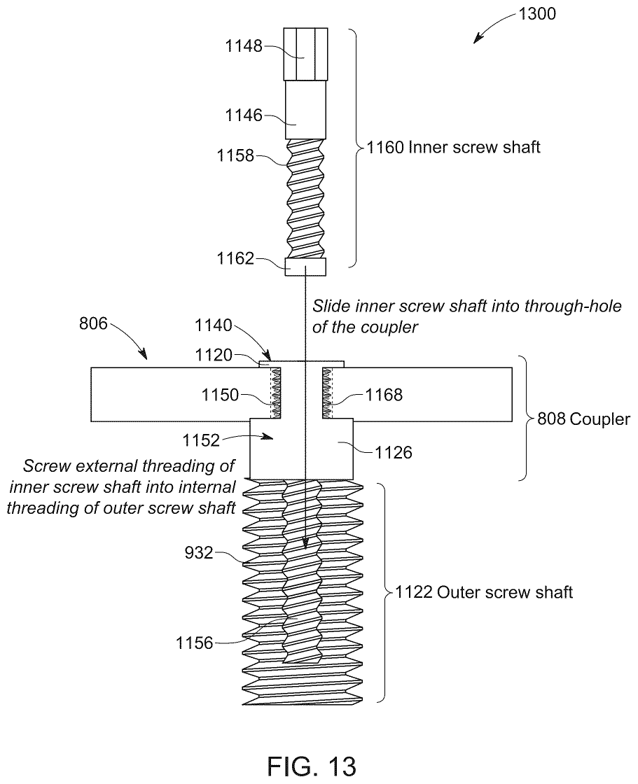

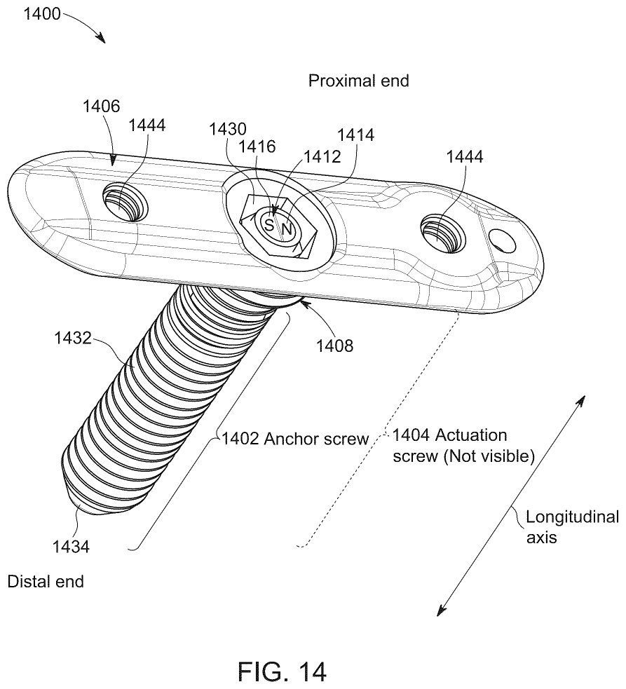

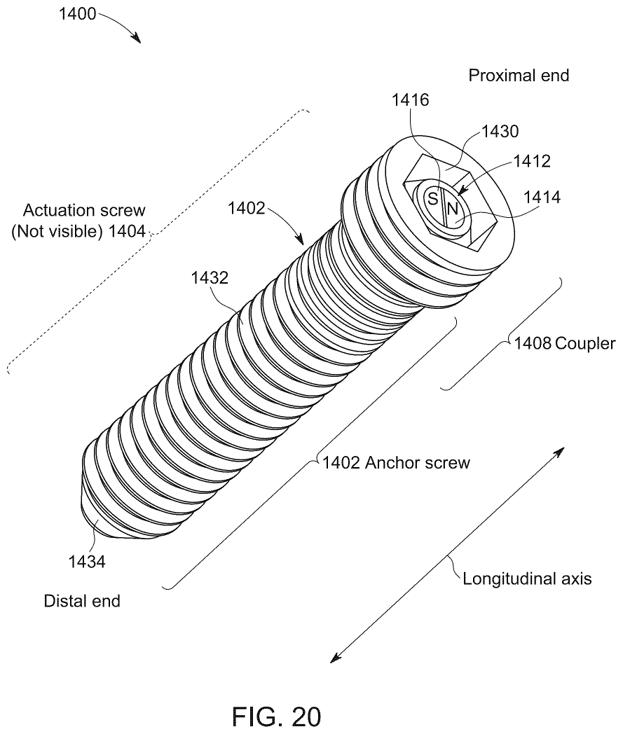

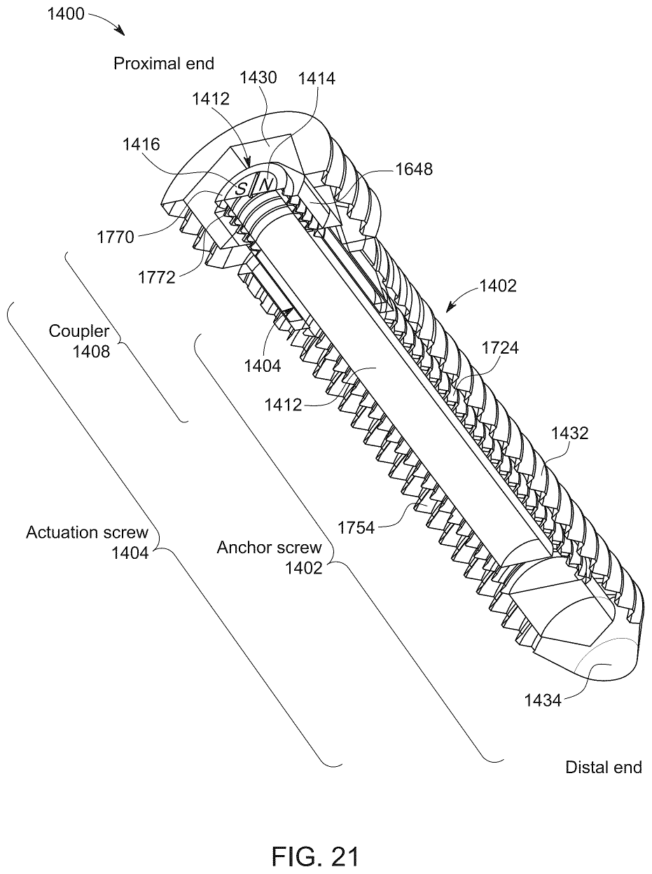

FIGS. 14-21 illustrate various views of a system 1400 for performing a distraction histogenesis surgical procedure. FIGS. 14-19 illustrate wherein the system 1400 includes a distraction plate, and FIGS. 20-21 illustrate wherein the system 1400 does not include the distraction plate. The system 1400 may be utilized with or without the distraction plate based on patient needs. In some cases, the system 1400 may be installed without the distraction plate, and then the distraction plate may be added later based on patient needs, and vice versa.

The system 1400 is configured to be magnetically coupled to an external magnet or electromagnet. Like the devices 100, 800 first described in connection with FIGS. 1 and 8, respectively, the system 1400 may be installed within a patient, and then distracted (i.e., expanded) or retracted (i.e., contracted) by establishing a magnetic coupling between the system 1400 and an external magnetic controller (EMC). Thus, the system 1400 may be installed within the patient and the patient's skin barrier may be fully closed over the system 1400. The system 1400 may still be distracted and retracted by establishing a magnetic coupling between the system 1400 and the external magnetic controller that is located external to the patient's skin barrier.

The system 1400 may be utilized in connection with a distraction plate (see, e.g., the distraction plate 806 first described in connection with FIG. 8) or may be utilized without the distraction plate. The decision on whether to include a distraction plate will be made by a healthcare provider depending upon the patient needs.

FIG. 14 is a perspective view of the system 1400 with the distraction plate 1406. The system 1400 includes an actuation screw 1404 fully retracted into an anchor screw 1402. Like the system 800 first described in connection with FIG. 8, the distraction plate 1406 is axially coupled to the actuation screw 1404 without also being rotationally coupled to the actuation screw 1404.

The system 1400 includes the anchor screw 1402 and the actuation screw 1404 configured to be disposed within the anchor screw 1402. The actuation screw 1404 comprises a magnetic core 1412 that comprising at least two poles, including at least a first pole 1414 and a second pole 1416. The magnetic core 1412 may comprise an elongated geometry such that each of the first pole 1414 and the second pole 1416 runs along a length of the magnetic core 1412. The magnetic core 1412 may comprise a length that is substantially equivalent to a length of the actuation screw 1404. The magnetic core 1412 may include more than two poles, and in some cases, this may be desirable to enable a machine or user to rotate the actuation screw 1404 (and thereby distract or retract the actuation screw 1404 from the anchor screw 1402) with increased granularity. The magnetic core 1412 may include two, four, six, eight, ten, or more poles in various implementations. Each of the various poles of the magnetic core 1412 may run the length of the magnetic core.

The actuation screw 1404 may include the magnetic core 1412 fully integrated into the actuation screw 1404 to form a singular indivisible element. In alternative implementations, the actuation screw 1404 may include a sidewall that defines a hollow interior, and the magnetic core 1412 may be disposed within the hollow interior. In such an implementation, the magnetic core 1412 may form an interference fit with the sidewall of the actuation screw 1404 and/or the magnetic core 1412 may include external threading that is configured to interface with internal threading of the anchor screw 1402.

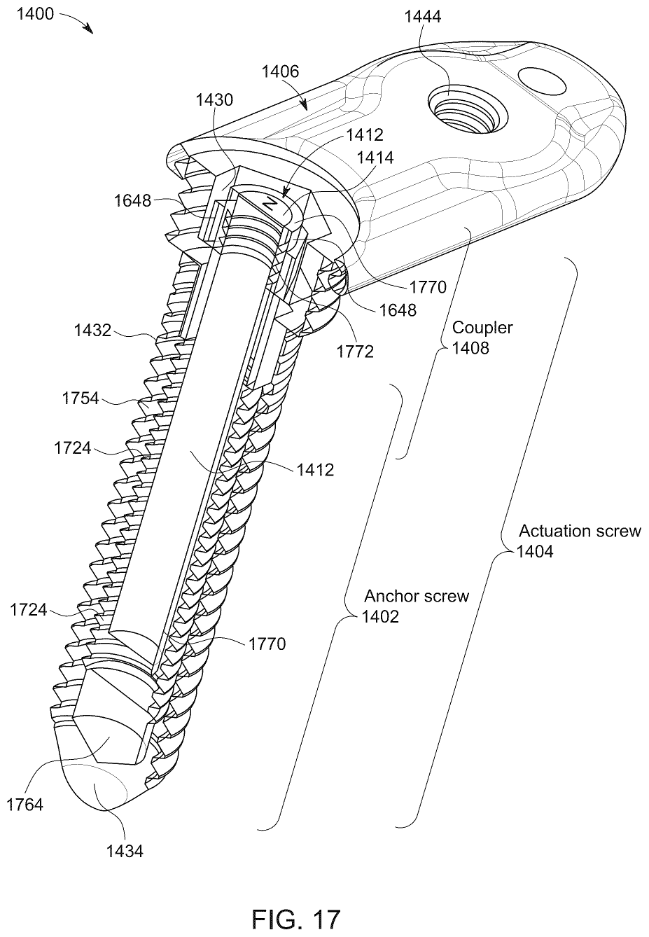

The anchor screw 1402 comprises a sidewall that defines a hollow interior. The anchor screw 1402 includes external threading 1432 attached to an exterior surface of the sidewall, and the external threading 1432 is configured to interface with a patient tissue substrate. The anchor screw 1402 may additionally include internal threading (see 1754 at FIG. 17) attached to an interior surface of the sidewall. The internal threading 1754 of the anchor screw 1402 is configured to interface with corresponding external threading (see 1724 at FIG. 17) of the actuation screw 1404.

The system 1400 includes the distraction plate 1406 that may be utilized to provide a larger surface for distracting a patient tissue. The distraction plate 1406 includes a central threaded hole configured to receive a coupler 1408. Like the coupler 808 described in connection with the system 800 first illustrated in FIG. 8, the coupler 1408 is configured to axially couple the distraction plate 1406 to the actuation screw 1404.

The coupler 1408 may include a socket interface 1430 comprising a polygonal cross-sectional geometry. The socket interface 1430 provides a means to remove the coupler 1408, actuation screw 1404, and anchor screw 1402 from a patient tissue substrate.

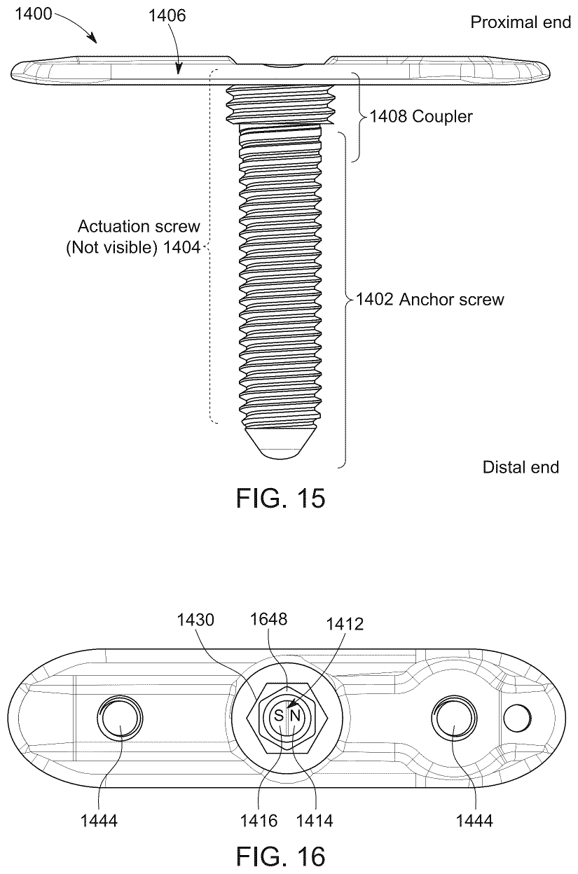

FIG. 15 is a straight-on side view of the system 1400 for performing a distraction histogenesis surgical procedure. FIG. 15 includes brackets illustrating the approximate lengths of the anchor screw 1402, actuation screw 1404, and coupler 1408. The actuation screw 1404 is disposed within the anchor screw 1402 and is thus not visible in a straight-on side view. The actuation screw 1404 may descend approximately to an end of the anchor screw 1402. The actuation screw 1404 may also be disposed through the coupler 1408 and the distraction plate 1406 and then terminate at approximately an upper surface of the distraction plate 1406.

FIG. 16 is a straight-on aerial top-down view of the system 1400 for performing a distraction histogenesis surgical procedure.

The socket interface 1430 of the coupler 1408 may include a polygonal cross-sectional geometry like the hexagonal geometry shown in FIG. 16. The socket interface 1430 is configured to interface with a corresponding driver having a polygonal cross-sectional geometry. The socket interface 1430 enables a user to unscrew the anchor screw 1402 from a patient tissue substrate.

The actuation screw 1404 may include a screw head 1648 that includes a polygonal cross-sectional geometry, such as a quadrilateral, pentagonal, hexagonal, and so forth, polygonal cross-sectional geometry. In the example illustrated herein, the screw head 1648 of the actuation screw 1404 includes a hexagonal driver head geometry. The socket interface 1430 may be sized such that the screw head 1648 may rotate within the socket interface 1430.

FIG. 17 is a cross-sectional perspective view of the system 1400 for performing a distraction histogenesis surgical procedure, wherein the actuation screw 1404 is fully retracted into the anchor screw 1402. The cross-sectional view of FIG. 17 enables viewing of the actuation screw 1404 that is fully screwed into the anchor screw 1402.

The actuation screw 1404 may include the screw head 1648 comprising a polygonal cross-sectional geometry. The screw head 1648 includes the polygonal cross-sectional geometry to enable a user or automated fixator adjustor to achieve sufficient torque when manually adjusting a position of the actuation screw 1404. Additionally, the polygonal cross-sectional geometry of the screw head 1648 enables a user to screw the entire system 1400 into a patient tissue substrate during an implantation surgery. Likewise, the polygonal cross-sectional geometry of the screw head 1648 aids in unscrewing the actuation screw 1404 during a removal surgery.

Like the actuation screw 104 first described in connection with FIG. 1, the actuation screw 1404 also includes external threading 1724 along a longitudinal shaft of an exterior of the actuation screw 1404. In some cases, the external threading 1724 may be attached directly to the magnetic core 1412. In other implementations, the external threading 1724 may be attached to a sidewall of the actuation screw 1404, and the magnetic core 1412 may be disposed within a hollow interior space defined by the sidewall of the actuation screw 1404. The external threading 1724 is configured to interface with corresponding internal threading 1754 of the anchor screw 1402. The actuation screw 1404 may include an actuation point 1764 disposed at a distal end of the actuation screw 1404. The actuation screw 1404 may be sized such that the actuation point 1764 is located near the anchor tip 1434 of the anchor screw 1402.

The actuation screw 1404 may be screwed into and out of the anchor screw 1402 by manually rotating the actuation screw 1404 with the screw head 1648. The actuation screw 1404 may additionally be screwed into and out of the anchor screw 1402 by rotating the magnetic core 1412. The magnetic core 1412 may be magnetically coupled to a corresponding magnet or electromagnet of an EMC. When the corresponding magnet of the EMC is rotated, the magnetic core 1412 of the actuation screw 1404 will also rotate. Thus, the EMC may be utilized to distract and retract the actuation screw 1404 without physically touching the actuation screw 1404. This enables a healthcare provider to implant the system 1400 and then close the patient's skin to reduce the likelihood of infection.

The magnetic core 1412 may include an elongated cylindrical geometry as shown in FIG. 17. The actuation screw 1404 may include an inner sheath 1770 configured to receive the magnetic core 1412. The inner sheath 1770 may be disposed within an outer sheath of the actuation screw 1404, wherein the outer sheath includes the external threading 1724 configured to engage with the internal threading 1754 of the anchor screw 1402. The inner sheath 1770 may include external threading 1772 that is configured to engage with corresponding internal threading of the screw head 1648 of the actuation screw 1404. The inner sheath 1770 may form a tight interference fit with the magnetic core 1412. In some cases, the magnetic core 1412 may be glued into or otherwise adhered to the inner sheath 1770.

The system 1400 may be implemented without the inner sheath 1770. In such an implementation, the magnetic core 1412 may be directly coupled to the actuation screw 1404 and then disposed within a hollow interior defined by the actuation screw 1404.

The magnetic core 1412 is coupled to the actuation screw 1404 such that rotation of the magnetic core 1412 causes corresponding rotation of the actuation screw 1404. When the magnetic core 1412 is magnetically coupled to a magnet or electromagnet of an EMC, the magnetic core 1412 may be rotated without directly contacting any component of the system 1400. The remote rotation of the magnetic core 1412 causes corresponding rotation of the actuation screw 1404 and may thereby cause the actuation screw 1404 to screw out of and into the anchor screw 1402. As described herein, the coupler 1408 serves as an anti-rotation assembly that enables the distraction plate 1406 to remain rotationally stationary when the actuation screw 1404 is screwed out of and into the anchor screw 1402.

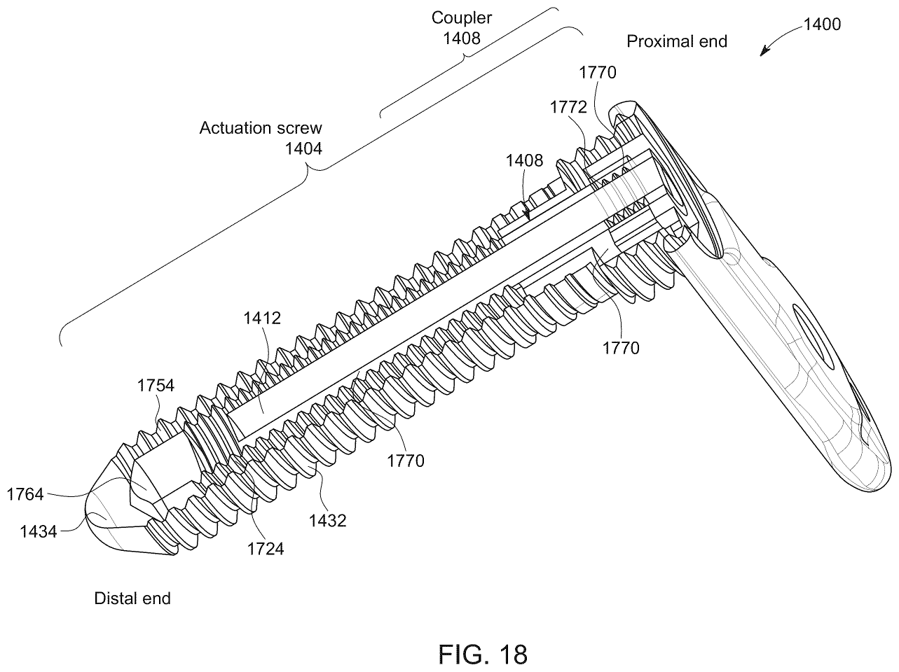

FIG. 18 is a cross-sectional perspective view of the system 1400 for performing a distraction histogenesis surgical procedure. The cross-sectional view of FIG. 18 enables further viewing of the inner sheath 1770 that may be disposed around the magnetic core 1412. The magnetic core 1412 may be arranged such that each pole of the magnetic core 1412 runs an entire longitudinal length of the magnetic core 1412.

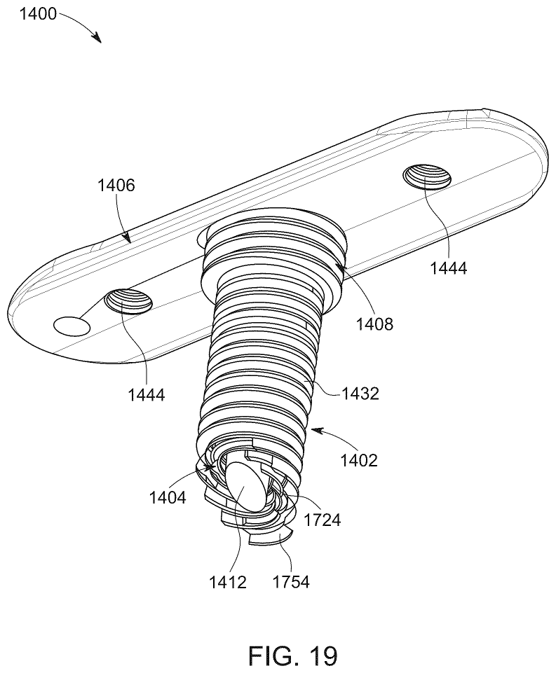

FIG. 19 is a cross-sectional perspective view of the system 1400 for performing a distraction histogenesis surgical procedure. The cross-sectional in FIG. 19 is cut through a portion of the magnetic core 1412, actuation screw 1404, and anchor screw 1402. FIG. 19 illustrates wherein the system 1400 does not include an inner sheath (see 1770 at FIG. 17) disposed around the magnetic core 1412. Instead, the magnetic core 1412 is disposed directly within a hollow interior defined by a sidewall of the actuation screw 1404.

FIGS. 20 and 21 illustrate views of the system 1400 without the distraction plate (see 1406 first illustrated in FIG. 14). FIG. 20 is a perspective view of the system 1400 and FIG. 21 is a cross-sectional perspective view of the system 1400. The system 1400 may be utilized with or without the distraction plate (see 1406) according to patient needs. In some cases, the system 1400 may be installed without the distraction plate, and then the distraction plate may be later added based on patient progression. In other cases, the system 1400 may be installed with the distraction plate, and then the distraction plate may be later removed based on patient progression.

The cross-sectional view of FIG. 21 illustrates wherein the inner sheath 1770 is disposed around the magnetic core 1412 only at a proximal end of the magnetic core 1412. The inner sheath 1770 may be glued to the magnetic core 1412 or otherwise adhered to the magnetic core 1412. The external threading 1772 of the inner sheath 1770 may then be utilized to couple the magnetic core to the screw head 1648 of the actuation screw 1404.

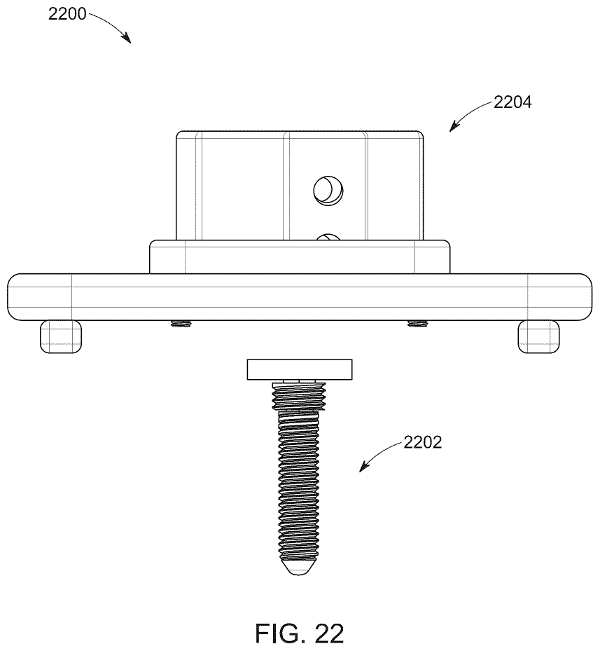

FIG. 22 is a straight-on side view of a system 2200 for performing a distraction histogenesis surgical procedure, and then actuating distraction and retraction of a device through a magnetic coupling. The system 2200 includes a distraction device 2202 for performing a distraction histogenesis surgical procedure. The system 2200 additionally includes an external magnetic controller (EMC) 2204 that forms a magnetic coupling with a magnet of the distraction device 2202.

In the example illustrated in FIG. 22, the distraction device 2202 is like the system 100 first described in connection with FIG. 1. However, the distraction device 2202 of the system 1400 may include a distraction plate and include components similar to the system 800 first described in connection with FIG. 8. Likewise, the system 1400 may include a distraction device 2202 including a magnetic core and include components similar to the system 1400 first described in connection with FIG. 14. The EMC 2204 may be utilized in connection with any of the magnetic devices described herein, including at least the devices 100, 800, 1400 first described in connection with FIGS. 1, 8, and 14, respectively.

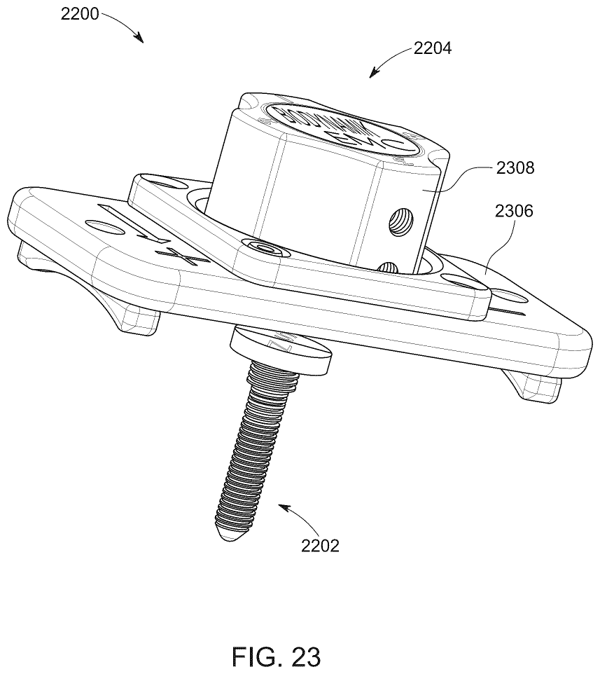

FIG. 23 is a perspective view of the system 2200 for performing a distraction histogenesis surgical procedure, and then actuating distraction and retraction of a device through a magnetic coupling. The EMC 2204 may include a bottom plate 2306 configured to rest against a patient. The EMC 2204 may additionally include a dial 2308 to enable a machine or human user to rotate an external magnet of the EMC 2204.

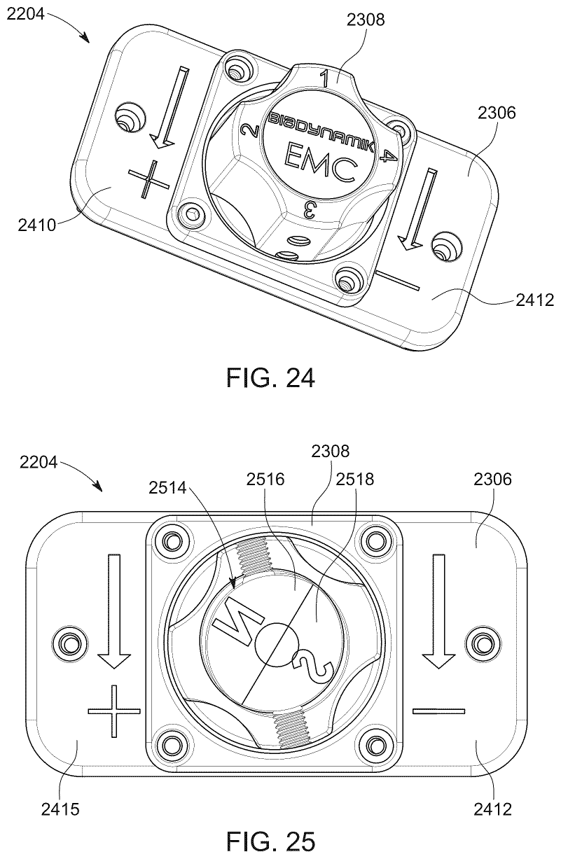

FIG. 24 is an overhead perspective view of the EMC 2204 for actuating distraction and retraction of a device through a magnetic coupling. The EMC 2204 may additionally include external markers, including a positive rotational marker 2410 indicating the direction to rotate the dial 2308 to distract an actuation screw, i.e., the direction to turn the dial 2308 to unscrew the actuation screw from the corresponding anchor screw. The EMC 2204 may additionally include a negative rotational marker 2412 indicating the direction to rotate the dial 2308 to retract the actuation screw, i.e., the direction to turn the dial 2308 to screw the actuation screw further into the corresponding anchor screw.

FIG. 25 is a cross-sectional aerial top-down view of the EMC 2204 for actuating distraction and retraction of a device through a magnetic coupling. The EMC 2204 includes an external magnet 2514 that comprises at least two poles having opposite polarity. As described herein, the external magnet 2514 is described as being “external” because it may be located external to patient tissue and still form a magnetic coupling with a magnetic device implanted inside the patient.

The external magnet 2514 includes at least a first pole 2516 and a second pole 2518 that are located adjacent to one another. The external magnet 2514 may include additional poles, including for example, four poles, six poles, eight poles, and so forth. In some cases, it may be desirable to include a higher quantity of poles to provide increased granularity when rotating the external magnet 2514.

The quantity of poles of the EMC 2204 may match the quantity of poles in a corresponding magnet of a device for performing a distraction histogenesis surgical procedure (see, e.g., the plate magnet 112 first described in connection with FIG. 1; the plate magnet 912 first described in connection with FIG. 9; or the magnetic core 1412 first described in connection with FIG. 14). The poles of the magnet of the device (see, e.g., 100, 800, or 1400) may alternate in polarity like pie slices around the magnet. Likewise, the poles of the external magnet 2514 of the EMC 2204 may alternate in polarity like pie slices around the external magnet 2514.

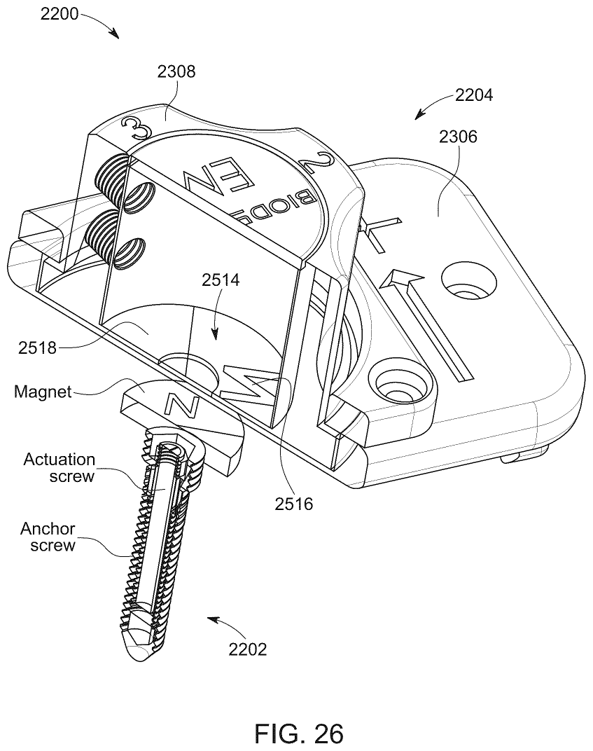

FIG. 26 is a cross-sectional perspective view of the system 2200 for performing a distraction histogenesis surgical procedure, and then actuating distraction and retraction of a device through a magnetic coupling. As shown in FIG. 26, a magnetic coupling is formed between the external magnet 2514 of the EMC 2204 and a corresponding magnet of the distraction device 2202.

In the example implementation illustrated in FIG. 26, the magnet of the distraction device 2202 includes two poles, including a North pole and a South pole. Likewise, the external magnet 2514 of the EMC 2204 includes two poles, including a North pole and a South pole. The opposite polarities attract between the magnet of the distraction device 2202 and the external magnet 2514 of the EMC 2204. This forms a magnetic coupling between the distraction device 2202 and the EMC 2204 that may be established even through numerous layers of intervening materials. The magnetic coupling may be sufficiently strong to be maintained through material layers such as periosteum, fat, fascia, skin, fabric, medical bandaging, and so forth.

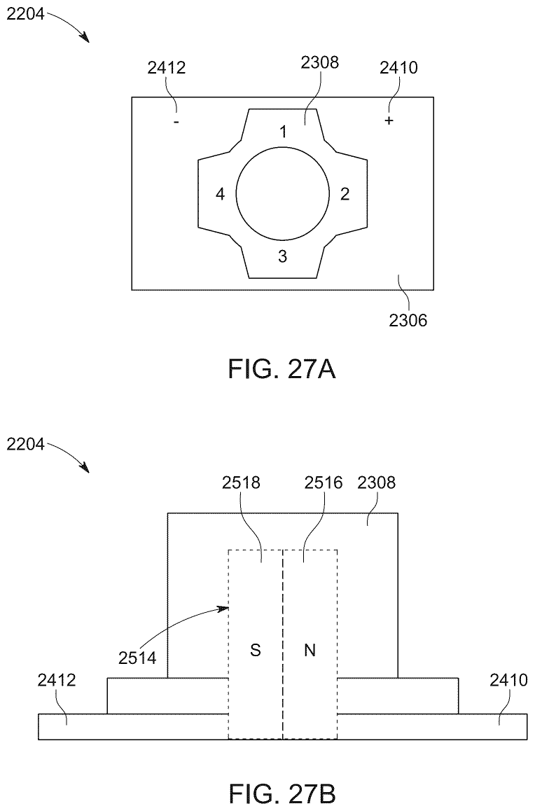

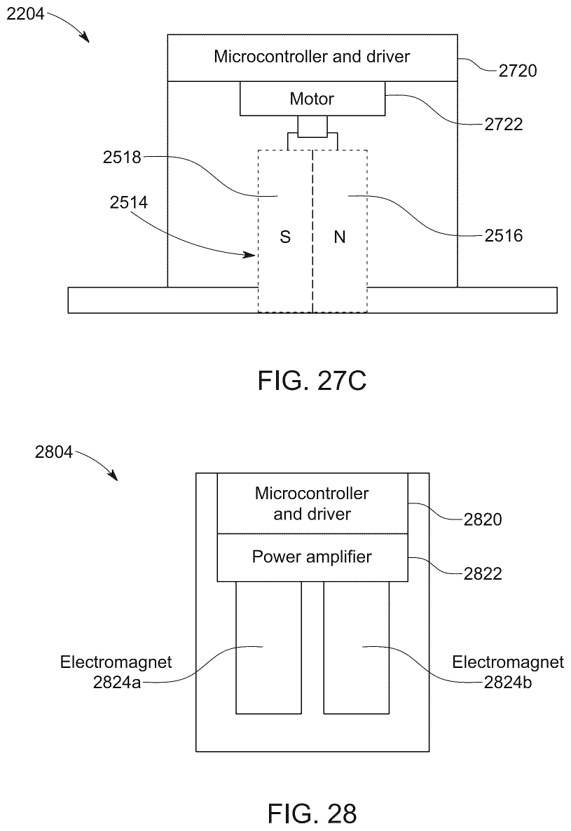

FIGS. 27A-27C are schematic illustrations of an EMC 2204 including an external magnet 2514. The EMC 2204 may be manually actuated by a user or automatically actuated by an electronic controller in communication with a motor.

FIG. 27A is a schematic illustration of an aerial top-down view of the EMC 2204. The EMC 2204 is configured to be magnetically coupled to the systems described herein, including any of the devices 100, 800, 1400 first described in connection with FIGS. 1, 8, and 14, respectively.

The EMC 2204 incudes a housing that comprises the dial 2308 that may be rotated clockwise and counterclockwise. In the example illustrated in FIG. 27, the dial 2308 may be rotated clockwise to rotate in a positive 2410 direction, which would further distract an actuation screw, and thus unscrew the actuation screw from a corresponding anchor screw to lift the actuation screw away from the anchor screw. The dial 2308 may be rotated counterclockwise in a negative 2412 direction, which would retract the actuation screw, and thus further screw the actuation screw into the corresponding anchor screw.

FIG. 27B is a schematic illustration of a straight-on cross-sectional view of the EMC 2204. The EMC 2204 includes the external magnet 2514 that includes a first polarity 2518 and a second polarity 2518. Like the magnet of an actuation screw, the external magnet 2514 is arranged such that each of the first polarity 2516 and the second polarity 2518 are located at the ends of the external magnet 2514. The external magnet 2514 may include a cylindrical geometry as shown in FIG. 27B, and in this implementation, the first polarity 2516 and the second polarity 2518 run the entire length of cylindrical geometry.

FIG. 27C is a schematic illustration of a straight-on cross-sectional view of the EMC 2204, wherein the EMC further includes a microcontroller and driver 2720 that is in electronic communication with a motor 2722. The motor 2722 is mechanically coupled to the external magnet 2514 such that rotation of the motor 2722 may cause rotation of the external magnet 2514.

FIG. 28 is a schematic illustration of a straight-on cross-sectional view of an EMC 2804 that includes one or more electromagnets 2824a, 2824b. The EMC 2804 may include a microcontroller and driver 2820 in electronic communication with the electromagnets 2824a, 2824b. The EMC 2804 may additionally include a power amplifier 2822 in electronic communication with the electromagnets 2824a, 2824b and the microcontroller and driver 2820, wherein the power amplifier 2822 powers a coil of the electromagnets 2824a, 2824b.

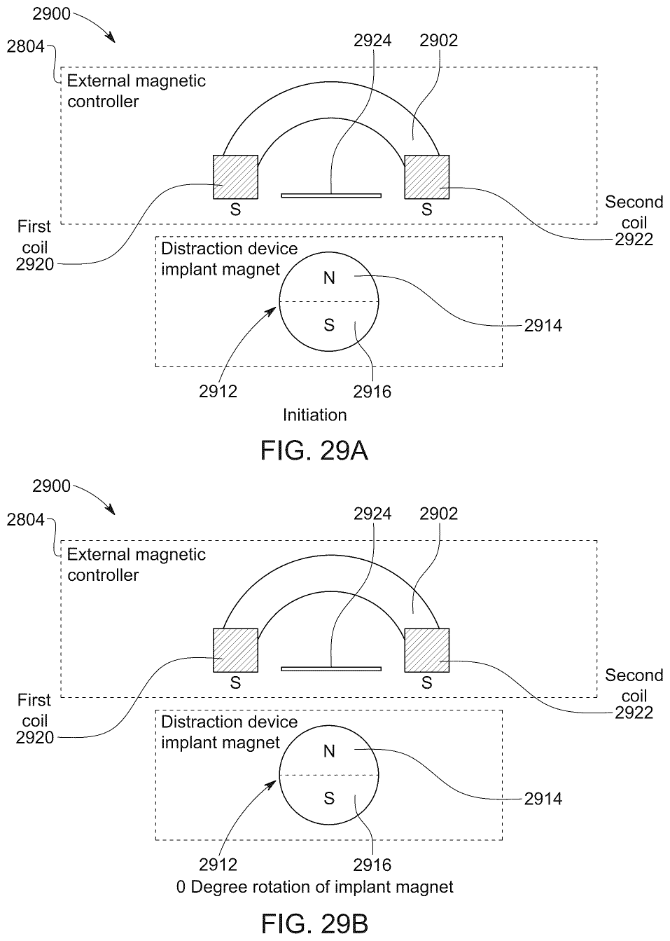

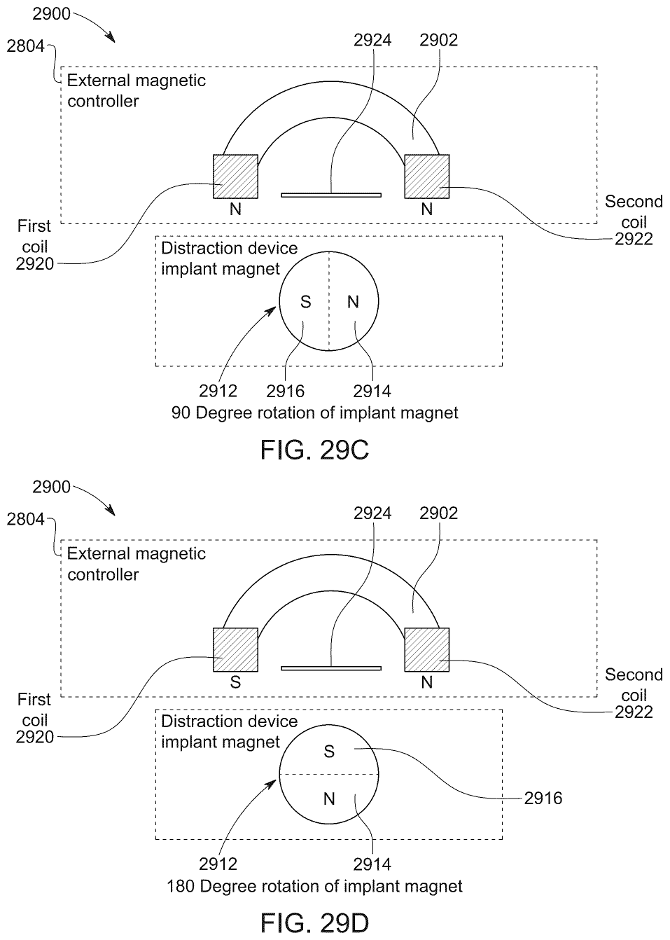

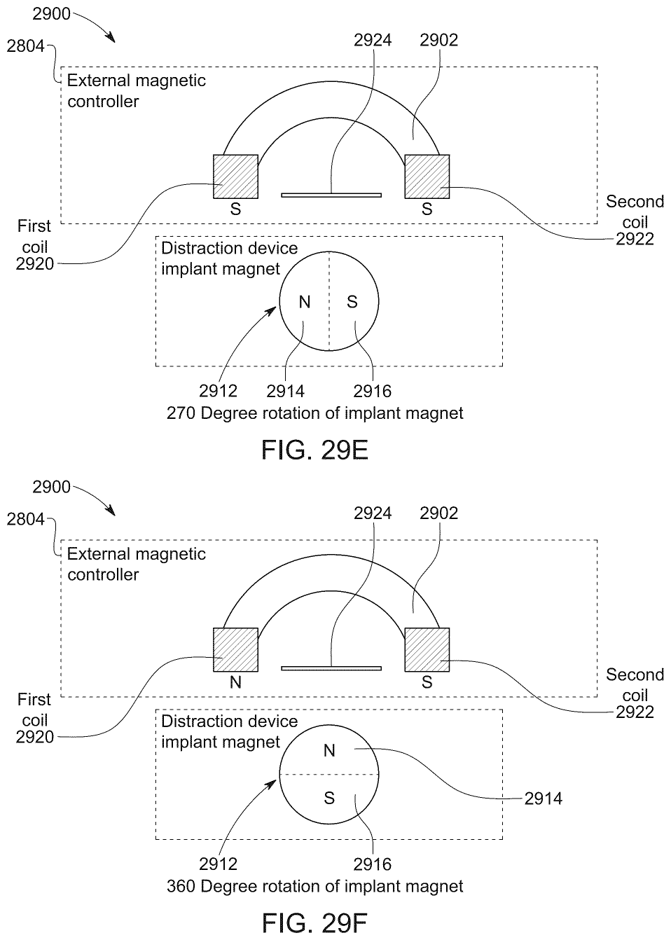

FIGS. 29A-29F are schematic illustrations of a system 2900 for driving an implant magnet 2912 of a distraction device with an EMC 2804 that includes electromagnets. FIGS. 29A-29F include a schematic illustration of a cross-sectional straight-on side view of the EMC 2804, and additionally include an aerial top-down view of the implant magnet 2912 of the distraction device to provide context to the current rotational position of the implant magnet 2912.

The system 2900 includes the implant magnet 2912, which is a component of a distraction device and is implanted in a patient. The implant magnet 2912 may include the plate magnet 112 of the distraction device first illustrated in connection with FIG. 1. The implant magnet 2912 may include the plate magnet 912 of the distraction device first illustrated in connection with FIG. 8. The implant magnet 2912 may include the magnetic core 1412 of the distraction device first illustrated in connection with FIG. 14. The implant magnet 2912 includes at least a first pole 2914 and a second pole 2916. The implant magnet 2912 may include more than two poles, and may specifically include four, six, eight, or more poles.

The system 2900 is arranged such that the EMC 2804 includes a two-pole electromagnet 2902 comprising a first coil 2920 and a second coil 2922. The EMC 2804 includes a Hall effect sensor or Hall effect array 2924 configured to track a current orientation of the implant magnet 2912 of the distraction device. The system 2900 may be implemented with a two pole electromagnet 2902 that is connected as shown in FIGS. 29A-29F. The system 2900 may alternatively be implemented with a two pole electromagnet 2902 that comprises two separate (non-connected) electromagnets.

The schematic illustrations of FIGS. 29A-29F describe a principle of traveling waves that enable granular rotation of the implant magnet 2912 by engaging the two-pole electromagnet 2902 of the EMC 2804. The two-pole electromagnet 2902 may be arranged as an array around the implant magnet 2912 when the EMC 2804 is placed on the patient's skin to form a magnetic coupling with the implant magnet 2912.

FIG. 29A illustrates an initiation orientation for the system 2900. The two-pole electromagnet 2902 includes the first coil 2920 and the second coil 2922. The EMC 2804 further includes the Hall effect sensor or Hall effect array 2924 to track the current rotational orientation of the implant magnet 2912. The first coil 2920 and the second coil 2922 are located at approximately the junction between the first pole 2914 and the second pole 2916 of the implant magnet 2912.

FIG. 29B illustrates a zero degree rotation orientation for the system 2900, wherein all components of the system are in the same configuration as described in connection with the initiation orientation of FIG. 29A.

FIG. 29C illustrates a 90 degree rotation orientation for the system 2900. The implant magnet 2912 has rotated 90 degrees relative to the initiation orientation illustrated in FIG. 29A. The second coil 2922 is engaged.

FIG. 29D illustrates a 180 degree rotation orientation for the system 2900. The implant magnet 2912 has rotated 180 degrees relative to the initiation orientation illustrated in FIG. 29A. The second coil 2922 is engaged.

FIG. 29E illustrates a 270 degree rotation orientation for the system 2900. The implant magnet 2912 has rotated 270 degrees relative to the initiation orientation illustrated in FIG. 29A. The second coil 2922 is engaged.

FIG. 29F illustrates a 360 degree rotation orientation for the system 2900. The implant magnet 2912 has the same rotational position as the initiation orientation illustrated in FIG. 29A. The first coil 2920 and the second coil 2922 are engaged.

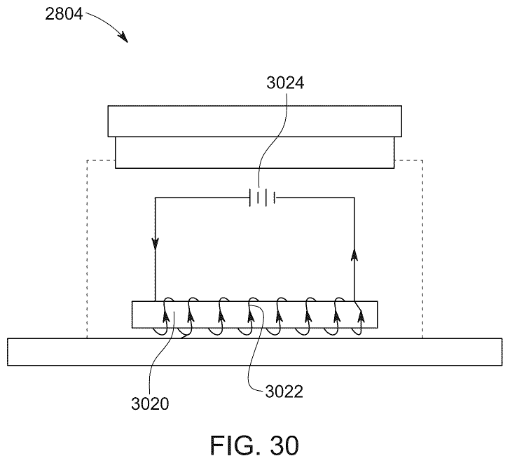

FIG. 30 is a schematic illustration of a straight-on cross-sectional view of an EMC 2804 comprising an electromagnet. The EMC 2804 may be utilized in connection with any of the devices described herein, including any of the devices 100, 800, or 1400 first described in connection with any of FIG. 1, 8, or 14, respectively. The EMC 2804 may be used in connection with any of the methods, systems, or described herein. The electromagnet-based EMC 2804 illustrated in FIG. 30 is interchangeable with the magnet-based EMC 2204 first illustrated in FIG. 22, as long as the electromagnet-based EMC 2804 is suitable for use based on a particular patient's health history.

The EMC 2804 may include housing components similar or identical to the housing components associated with the magnet-based EMC 2204 first illustrated in connection with FIG. 22. The electromagnet-based EMC 2804 includes an electromagnet comprising a rod 3020, a coil 3022 wrapped around the rod 3020, and a battery 3024. The coil 3022 may be driven by a power amplifier and controller by a microcontroller.

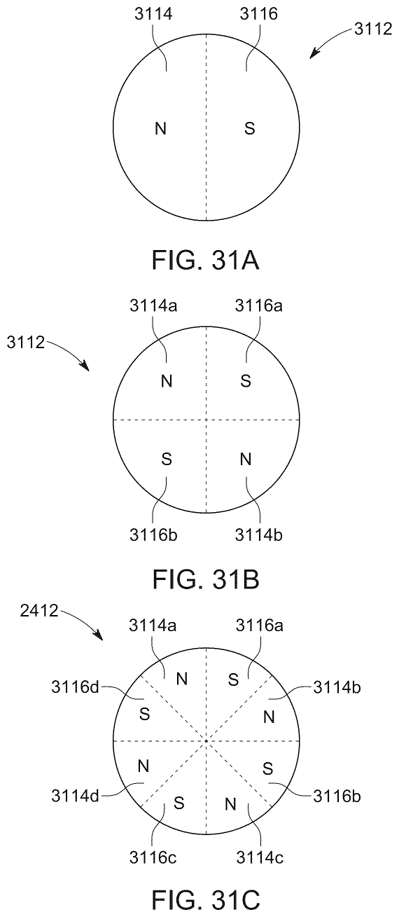

FIGS. 31A-31C are schematic cross-sectional aerial top-down views of exemplary magnets that may be utilized in connection with any of the devices described herein, including, for example, the devices 100, 800, or 1400 first described in connection with FIG. 1, 8, or 14, respectively. FIGS. 31A-31C each illustrate an aerial top-down view of a top of a magnet 3112 or cross-sectional slice of the magnet 3112.

FIG. 31A illustrate an example wherein the magnet 3112 includes two poles, including a first pole 3114 including a first polarity and a second pole 3116 including a second polarity. The first polarity is opposite to the second polarity. The poles 3114, 3116 are adjacent to one another in the cross-sectional slice such that each of the poles 3114, 3116 comprises a half-circle cross-sectional geometry.

FIG. 31B illustrates an example wherein the magnet 3112 includes four poles, including two of a first pole 3114a, 3114b each having a first polarity. The magnet 3112 further includes two of a second pole 3116a, 3116b each having a second polarity that is opposite to the first polarity. The poles are arranged in an alternating fashion such that any of the first pole 3114a, 3114b is adjacent to two of the second pole 3116a, 3116b. The poles are further arranged such that each of the poles 3114a, 3114b, 3116a, 3116b comprises a circular sector (pie slice) cross-sectional geometry.

FIG. 31C illustrates an example wherein the magnet 3112 includes eight poles, including four of a first pole 3114a, 3114b, 3114c, 3114d each having a first polarity. The magnet 3112 further includes four of a second pole 3116a, 3116b, 3116c, 3116d each having a second polarity that is opposite to the first polarity. The poles are arranged in an alternating fashion such that any of the first pole 3114a, 3114b, 3114c, 3114d is adjacent to two of the second pole. Likewise, any of the second pole 3116a, 3116b, 3116c, 3116d is adjacent to two of the first pole. The poles are further arranged such that each of the poles 3114a-3114d, 3116a-3116d comprises a circular sector (pie slice) cross-sectional geometry.

The devices 100, 800, 1400 described herein may be implemented with a magnet that comprises a plurality of a first pole having a first polarity, and a plurality of second pole having a second polarity that is opposite to the first polarity. Additionally, any of the external magnetic controllers 1404 described herein may include an external magnet that comprises a plurality of a first pole having a first polarity, and a plurality of second pole having a second polarity that is opposite to the first polarity. The systems described herein may be implemented such that the magnet of the distraction device includes the same quantity of poles as the external magnet of the external magnetic controller. In some cases, it may be desirable to include a greater quantity of poles to enable increased granularity when rotating the external magnet of the EMC, which causes synchronous rotation of the magnet of the distraction device, and which further causes synchronous rotation of the actuation screw of the distraction device.

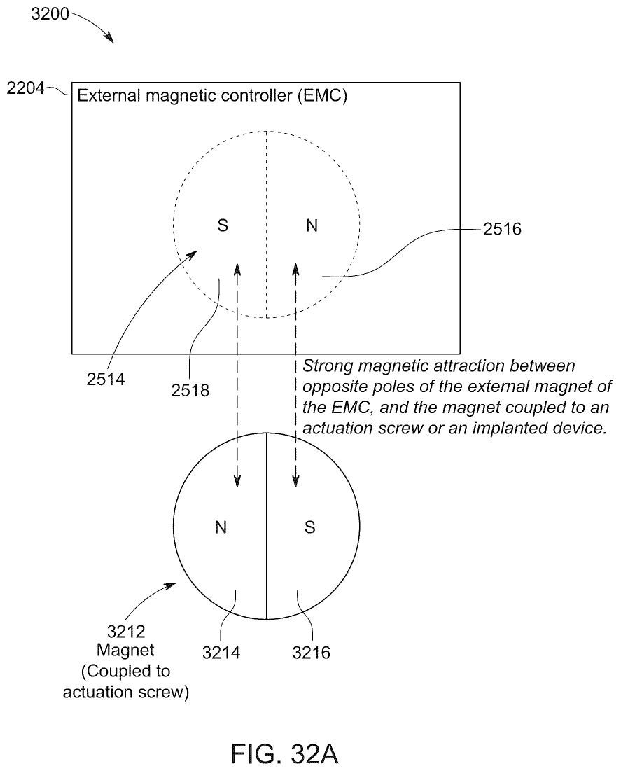

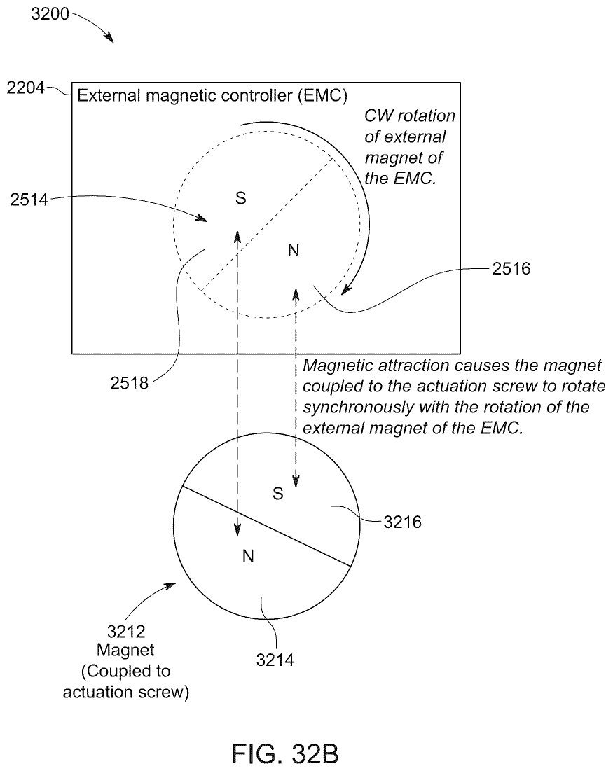

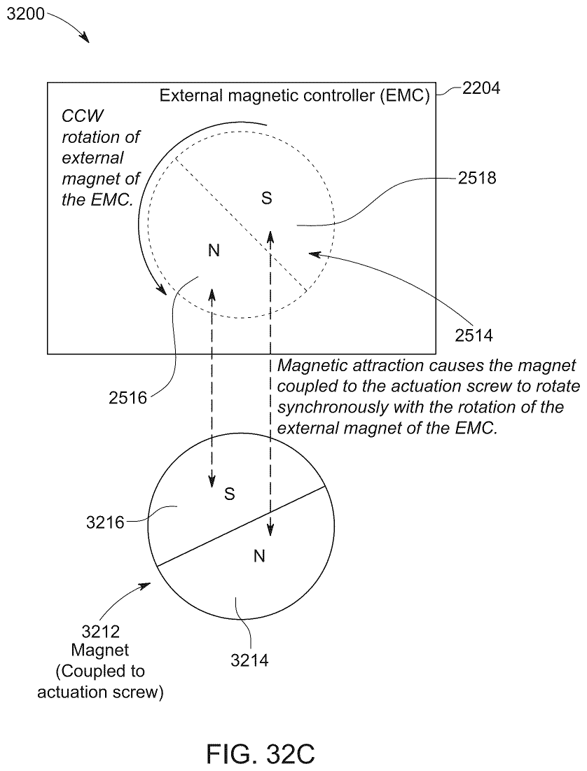

FIGS. 32A-32C are schematic illustrations depicting the magnetic coupling between a magnet 3212 of an actuation screw (see, e.g., 104, 804, 1404), and an external magnet 2514 of an EMC 2204. As shown in FIG. 32A, a first polarity 3214 of the magnet 3212 of the actuation screw establishes a magnetic attraction with the second polarity 2518 of the external magnet 2514. In the example illustrated in FIGS. 32A-32C, the first polarity 3214 of the magnet 3212 is a North polarity, and the second polarity 2518 of the external magnet 2514 is a South polarity. Similarly, the second polarity 3216 of the magnet 3212 establishes a magnetic attraction with the first polarity 2516 of the external magnet 2514. In the example illustrated in FIGS. 32A-32C, the second polarity 3216 of the magnet 3212 is a South polarity, and the first polarity 2516 of the external magnet 2514 is a North polarity. It should be appreciated that the polarities may be altered, and the polarities illustrated in FIGS. 32A-32B are exemplary only.

As shown in FIG. 32B, rotation of the external magnet 2514 of the EMC 2204 in a clockwise direction causes synchronous rotation of the magnet 3212 of the actuation screw due to the magnetic attraction of the corresponding magnetic poles. A patient or healthcare provider may thus place the EMC 2204 externally on the patient, establish the magnetic attraction between the EMC 2204 and the magnet 3212, and then distract or retract the actuation screw by rotating the external magnet 2514 of the EMC 2204.

As shown in FIG. 32C, rotation of the external magnet 2514 of the EMC 2204 in a counterclockwise direction causes synchronous rotation of the magnet 3212 of the actuation screw due to the magnetic attraction of the corresponding magnetic poles. A patient or healthcare provider may thus place the EMC 2204 externally on the patient, establish the magnetic attraction between the EMC 2204 and the magnet 3212, and then distract or retract the actuation screw by rotating the external magnet 2514 of the EMC 2204.

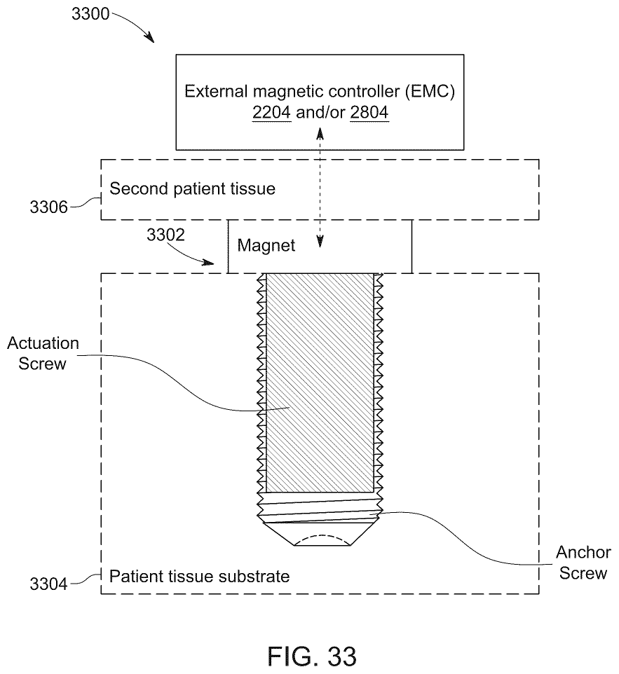

FIG. 33 is a schematic illustration of a distraction configuration 3300 for distracting or retracting a patient tissue after installation of a device 3302 for performing a distraction histogenesis surgical procedure. The device 3302 may include any of the suitable devices described herein, including, for example, any of the devices 100, 800, 1400 first described in any of FIG. 1, 8, or 14, respectively.

The distraction configuration 3300 includes an anchor screw anchored into a patient tissue substrate 3304. In the case of a periosteal distraction procedure, the patient tissue substrate 3304 may include bone tissue. However, the distraction configuration 3302 may be implemented on other patient tissue types. The distraction configuration 3300 includes a magnet of an actuation screw disposed in between the patient tissue substrate 3304 and a second patient tissue 3306. In the case of the periosteal distraction procedure, the patient tissue substrate 3304 may include the bone tissue, and the second patient tissue 3306 may include a periosteum layer. The distraction configuration 3300 includes the EMC 2204, 2804 located external to at least the second patient tissue 3306. In the case of a periosteal distraction procedure, the EMC 2204, 2804 may be located externally to the periosteum layer and may additionally be located externally to other layers such as connective tissue, fat, muscle, skin, and so forth.

The systems, methods, and devices described herein enable a healthcare provider to install a device for performing a distraction histogenesis procedure, and then fully close up the patient's skin barrier after installation. A user may then distract or retract the system by forming a magnetic attraction between the magnet coupled to an actuation screw, and the external magnet of the EMC 2204, 2804. This magnetic attraction enables the user to distract or retract the actuation screw over time without reopening the patient's skin barrier. This reduces the likelihood of infection and significantly improve patient outcomes.

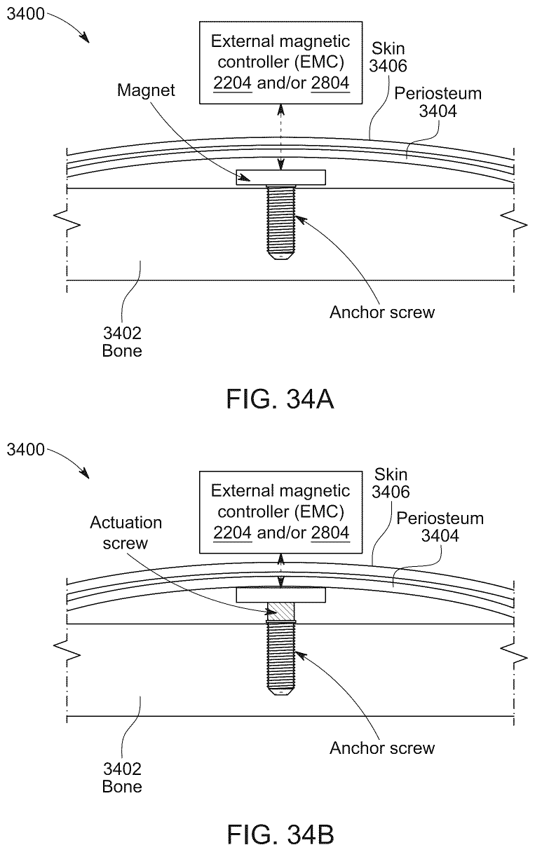

FIGS. 34A and 34B are schematic illustrations of a distraction configuration 3400 for performing a periosteal distraction surgical procedure on patient tissue. FIG. 34A illustrates wherein a device is fully retracted. FIG. 34B illustrates wherein the device is partially distracted. FIGS. 34A-34B specifically illustrate wherein the distraction device does not include a distraction plate.

The systems, methods, and devices described herein may be utilized to perform various distraction histogenesis procedures on various tissue types, including a variety of soft tissues. The systems, methods, and devices described herein may specifically be utilized to perform a periosteal distraction surgical procedure as depicted in FIGS. 34A-34B. The distraction configuration 3400 for a periosteal distraction procedure includes anchoring the anchor screw into a bone 3402 of a patient, and then fully screwing the actuation screw into the anchor screw. A periosteum 3404 may be pulled over a magnet coupled to the actuation screw, and the skin 3406 of the patient may be fully sutured shut to reduce the likelihood of infection. After the distraction device is installed, a user may place the EMC 2204, 2804 on the patient's skin over the actuation screw. When the EMC 2204, 2804 forms a magnetic attraction with the magnet coupled to the actuation screw, the user may rotate the dial of the EMC 2204, 2804 to synchronously rotate the actuation screw within the anchor screw. The user may thereby distract or retract the actuation screw within the anchor screw. As shown in FIG. 34B, when the actuation screw is partially distracted from the anchor screw, the periosteum 3404 and skin 3406 layers of the patient will be lifted away from the bone 3402 of the patient. This may trigger regeneration of bone tissue.

The distraction configuration 3400 may be performed utilizing any of the devices described herein. The distraction configuration 3400 may be performed utilizing the system 100 first described in connection with FIG. 1, wherein an anchor screw 102 is configured to receive an actuation screw 104 that includes a plate magnet 112 attached to the actuation screw 104. The plate magnet 112 may serve to stretch a second tissue layer up and away from the patient tissue substrate.

The distraction configuration 3400 may be performed utilizing the system 1400 first described in connection with FIG. 14, and specifically the implementation without the distraction plate as illustrated in FIGS. 20-21, wherein an anchor screw 1402 is configured to receive an actuation screw 1404 that is coupled to a magnetic core 1412. The magnetic core 1412 may serve to stretch a second tissue layer up and away from the patient tissue substrate.

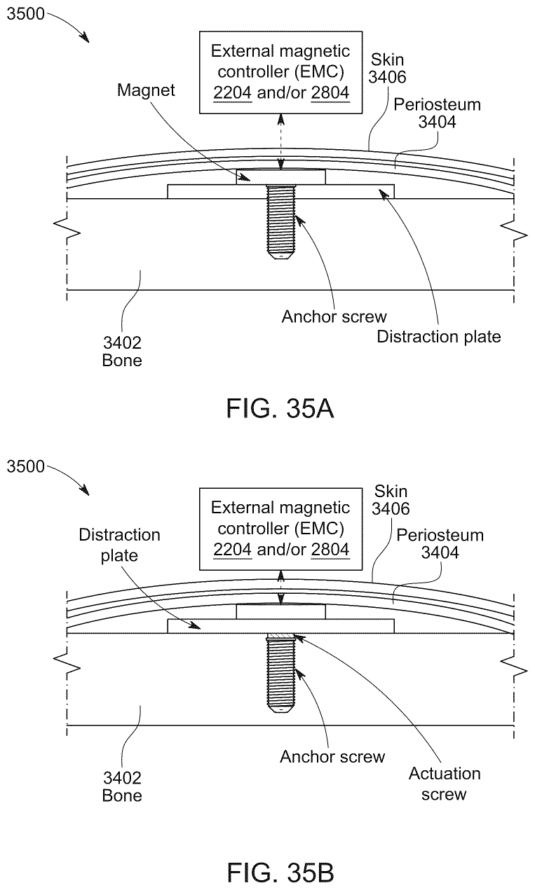

FIGS. 35A and 35B are schematic illustrations of a distraction configuration 3500 for performing a periosteal distraction surgical procedure on patient tissue. FIG. 35A illustrates wherein a device is fully retracted. FIG. 35B illustrates wherein the device is partially distracted. FIGS. 35A-35B specifically illustrate wherein the distraction device includes a distraction plate.

The systems, methods, and devices described herein may be utilized to perform various distraction histogenesis procedures on various tissue types, including a variety of soft tissues. The systems, methods, and devices described herein may specifically be utilized to perform a periosteal distraction surgical procedure as depicted in FIGS. 35A-35B. The distraction configuration 3500 for a periosteal distraction procedure includes anchoring the anchor screw into a bone 3402 of a patient, and then fully screwing the actuation screw into the anchor screw. A periosteum 3404 may be pulled over a magnet coupled to the actuation screw, and the skin 3406 of the patient may be fully sutured shut to reduce the likelihood of infection. After the distraction device is installed, a user may place the EMC 2204, 2804 on the patient's skin over the actuation screw. When the EMC 2204, 2804 forms a magnetic attraction with the magnet coupled to the actuation screw, the user may rotate the dial of the EMC 2204, 2804 to synchronously rotate the actuation screw within the anchor screw. The user may thereby distract or retract the actuation screw within the anchor screw. As shown in FIG. 35B, when the actuation screw is partially distracted from the anchor screw, the periosteum 3404 and skin 3406 layers of the patient will be lifted away from the bone 3402 of the patient. This may trigger regeneration of bone tissue.

The distraction configuration 3500 may be performed utilizing the system 800 first described in connection with FIG. 8, wherein an anchor screw 802 is configured to receive an actuation screw 804 that is coupled to a plate magnet 812 and coupled to a distraction plate 806. The plate magnet 812 and the distraction plate 806 may each serve to stretch a second tissue layer up and away from the patient tissue substrate. In some cases, the distraction plate may be secured to an osteotomized tissue such that the osteotomized tissue is also distracted away from the bone 3402 when the distraction plate is distracted away from the bone 3402.

The distraction configuration 3500 may be performed utilizing the system 1400 first described in connection with FIG. 14, wherein an anchor screw 1402 is configured to receive an actuation screw 1404 that is coupled to a magnetic core 1412 and may optionally additionally be coupled to a distraction plate (not shown in FIGS. 14-15). The magnet 1412 and the distraction plate (if utilized) may each serve to stretch a second tissue layer up and away from the patient tissue substrate. In some cases, the distraction plate may be secured to an osteotomized tissue such that the osteotomized tissue is also distracted away from the bone 3402 when the distraction plate is distracted away from the bone 3402.

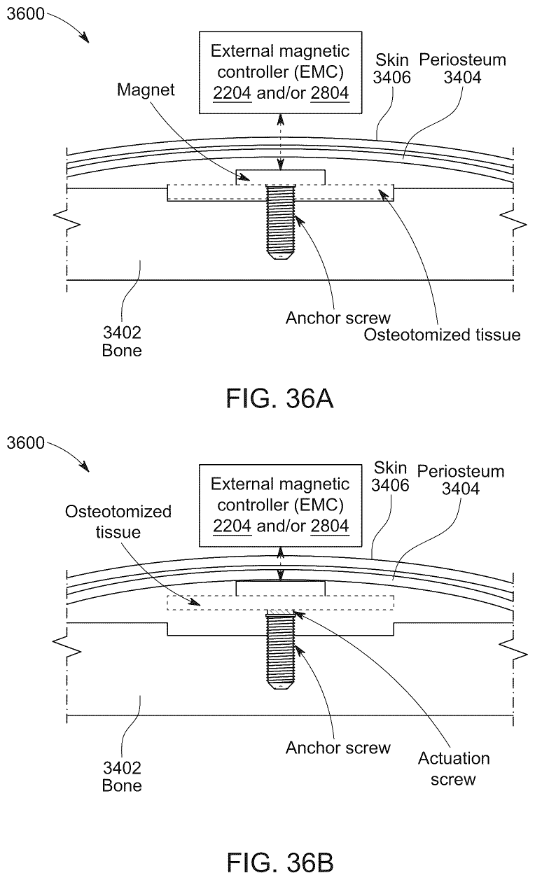

FIGS. 36A and 36B are schematic illustrations of a distraction configuration 3600 for performing an osteotomy distraction surgical procedure on patient tissue. FIG. 36A illustrates wherein a device is fully retracted. FIG. 36B illustrates wherein the device is partially distracted. The distraction configuration 3600 may be performed with or without a distraction plate. If the distraction plate is utilized, then the osteotomized tissue may be secured to the distraction plate. If the distraction plate is not utilized, then the osteotomized tissue may be secured to another component of the distraction device, such as the actuation screw or the coupler.

The systems, methods, and devices described herein may be utilized to perform various distraction histogenesis procedures on various tissue types, including a variety of soft tissues. The systems, methods, and devices described herein may specifically be utilized to perform an osteotomy distraction surgical procedure as depicted in FIGS. 36A-36B. The distraction configuration 3600 for an osteotomy distraction procedure includes anchoring the anchor screw into a bicortical bone 3402 of a patient, cutting out a portion of the nearside/proximal bone (i.e., the osteotomized tissue), anchoring a portion of the device to the osteotomized tissue, and fully screwing the actuation screw into the anchor screw. A periosteum 3404 may be pulled over a magnet coupled to the actuation screw, and the skin 3406 of the patient may be fully sutured shut to reduce the likelihood of infection. In another embodiment, the periosteum 3404 may be placed undeath the magnet coupled to the actuation screw. After the device is installed, a user may place the EMC 2204, 2804 on the patient's skin over the actuation screw. When the EMC 2204, 2804 forms a magnetic attraction with the magnet coupled to the actuation screw, the user may rotate the dial of the EMC 2204, 2804 to synchronously rotate the actuation screw within the anchor screw. The user may thereby distract or retract the actuation screw within the anchor screw. As shown in FIG. 36B, when the actuation screw is partially distracted from the anchor screw, the periosteum 3404 and skin 3406 layers of the patient will be lifted away from the bone 3406 of the patient, and the osteotomized tissue will likewise lift away from the anchoring bone tissue. This may trigger regeneration of bone, vascular or other soft tissue.

The distraction configuration 3600 may be performed utilizing the system 100 first described in connection with FIG. 1, wherein an anchor screw 102 is configured to receive an actuation screw 104 that includes a plate magnet 112 attached to the actuation screw 104. The plate magnet 112 may serve to stretch a second tissue layer up and away from the patient tissue substrate. The osteotomized bone tissue may be anchored to one or more of the plate magnet 112 or the actuation screw 104.

The distraction configuration 3600 may be performed utilizing the system 800 first described in connection with FIG. 8, wherein an anchor screw 802 is configured to receive an actuation screw 804 that is coupled to a plate magnet 812 and may optionally additionally be coupled to a distraction plate 806. The plate magnet 812 and the distraction plate 806 (if utilized) may each serve to stretch a second tissue layer up and away from the patient tissue substrate. The osteotomized tissue may be anchored to the distraction plate 806.

The distraction configuration 3600 may be performed utilizing the system 1400 first described in connection with FIG. 14, wherein an anchor screw 1402 is configured to receive an actuation screw 1404 that is coupled to a magnetic core 1412 and may optionally additionally be coupled to a distraction plate (not shown in FIGS. 14-15). The magnetic core 1412 and the distraction plate (if utilized) may each serve to stretch a second tissue layer up and away from the patient tissue substrate. The osteotomized tissue may be anchored to the distraction plate if utilized. The osteotomized tissue may be anchored to one or more of the coupler or the actuation screw if the distraction plate is not utilized.

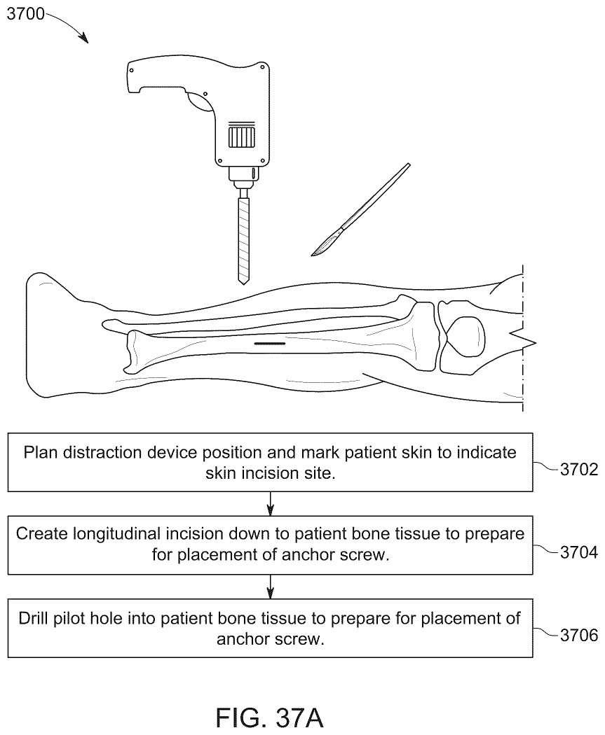

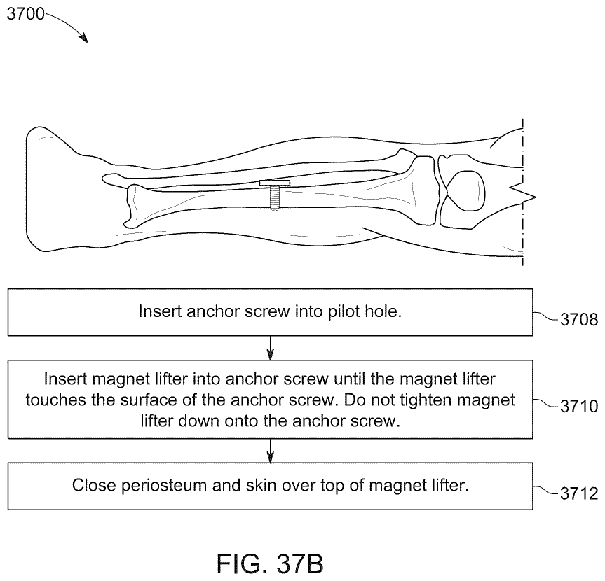

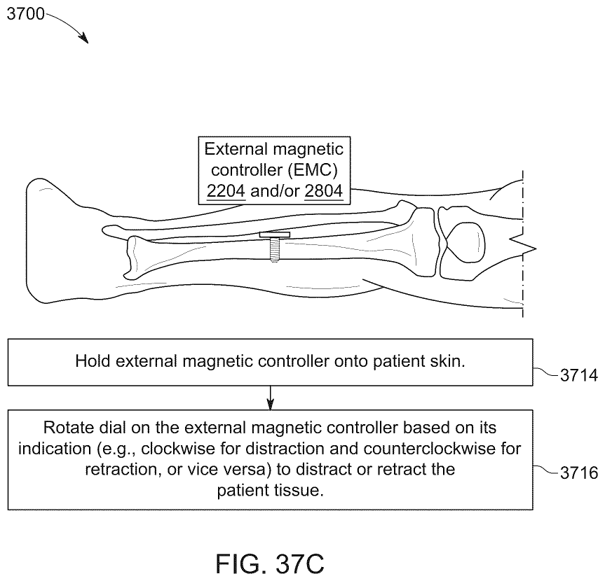

FIGS. 37A-37C are schematic flow chart diagrams of a method 3700 for performing a distraction histogenesis surgical procedure. The method 3700 described in FIGS. 37A-37C refers to steps for performing a periosteal distraction procedure to distract a periosteum layer away from a bone tissue layer. However, the method 3700 may be easily modified to perform similar steps on other tissue types.

The method 3700 includes planning at 3702 a distraction device position and marking patient skin to indicating skin incision site. The method 3700 includes creating at 3704 a longitudinal incision down to patient bone tissue to prepare for placement of anchor screw. The method 3700 includes drilling at 3706 a pilot hole into patient bone tissue to prepare for placement of anchor screw. The method 3700 includes inserting at 3708 the anchor screw into the pilot hole. The method 3700 includes inserting at 3710 an actuation screw (i.e., magnetic lifter) into the anchor screw until the actuation screw touches the surface of the anchor screw, and without tightening the actuation screw down onto the anchor screw. The method 3700 includes closing at 3712 the periosteum and skin over the top of the actuation screw. The method 3700 includes holding at 3714 the external magnetic controller on to the patient skin. The method 3700 includes rotating at 3718 the dial on the external magnetic controller based on its indication to distract or retract the patient tissue.

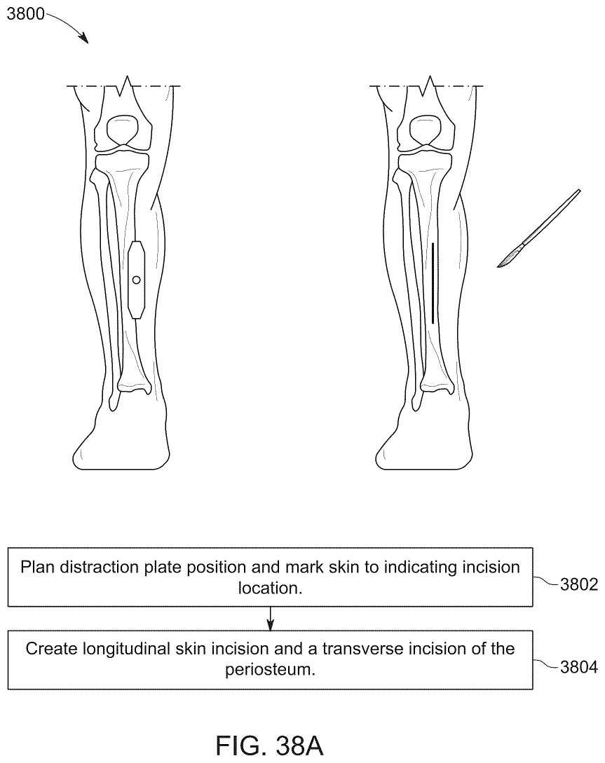

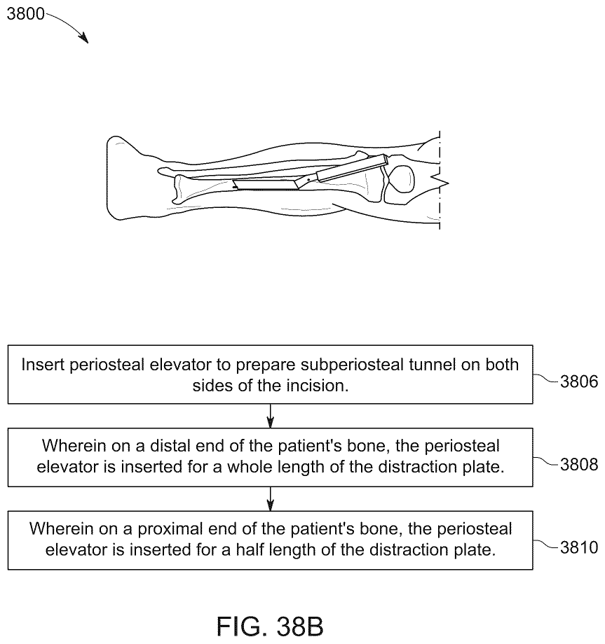

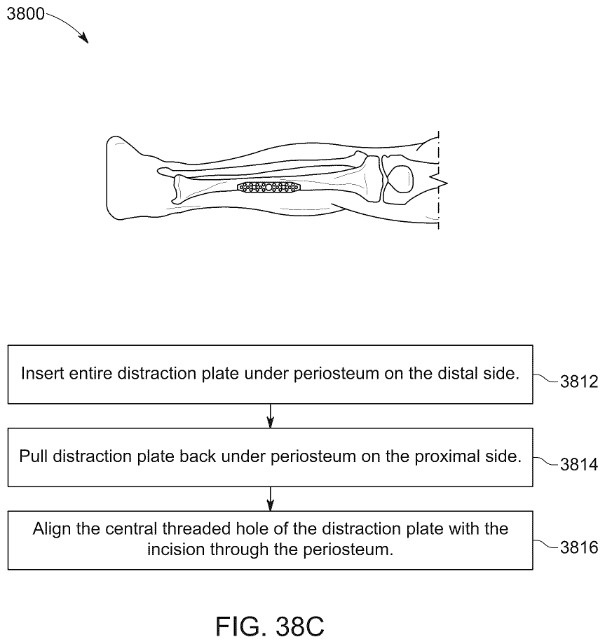

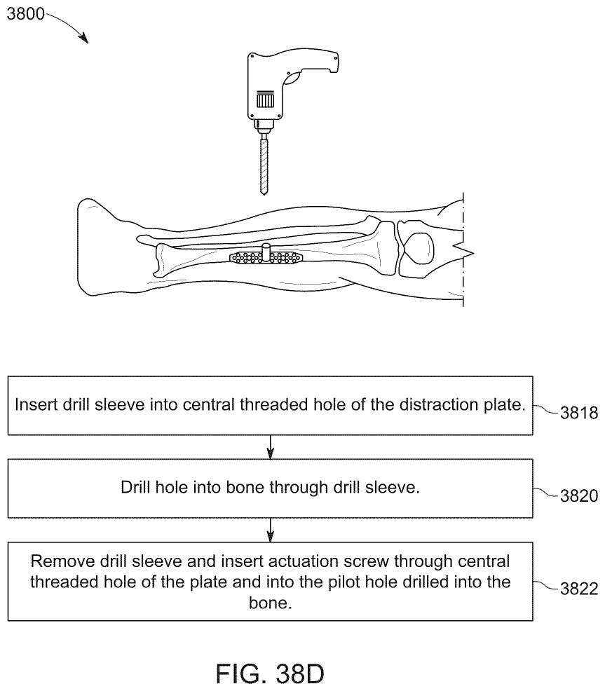

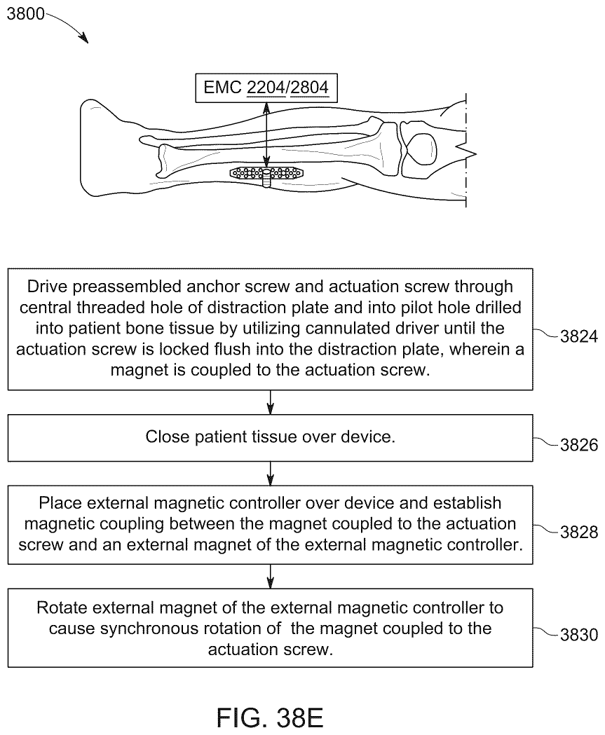

FIGS. 38A-38E are schematic block diagrams of a method 3800 for performing a distraction histogenesis surgical procedure utilizing the systems and devices described herein. In FIGS. 38A-38E, the method 3800 is being performed on a tibia bone of a patient as an exemplary use-case. It should be appreciated that the systems, methods, and devices described herein may be utilized on alternative bones and tissues as deemed appropriate. The systems described herein may be utilized to perform a periosteal distraction procedure as described in connection with FIGS. 38A-38E. The systems described herein may additionally be utilized to perform distraction histogenesis procedures on other tissue types.

The method 3800 may begin with planning a distraction plate position at 3802 and marking the patient's skin to indicate an incision location. This step is only necessary if the healthcare provider determines that a distraction plate would improve patient outcomes based on the patient's unique health circumstances. In some cases, the healthcare provider may determine that a distraction plate is not necessary, and that the patient may benefit from only the use of an anchor screw and actuation screw for distracting and retracting the patient's tissue.

The method 3800 includes creating at 3804 a longitudinal or transverse skin incision and a transverse incision of the patient's periosteum layer. The method 3800 includes inserting at 3806 a periosteal elevator to prepare subperiosteal tunnel on both sides of the incision. The method 3800 is such that on a distal end of the patient's bone, the periosteal elevator is inserted for a whole length of the distraction plate. The method 3800 is such that on a proximal end of the patient's bone, the periosteal elevator is inserted for a half-length of the distraction plate. The method 3800 includes inserting at 3812 the entire distraction plate under the periosteum on the distal side. The method 3800 includes pulling at 3814 the distraction plate back under the periosteum on the proximal side. The method 3800 include aligning at 3816 the central threaded hole of the distraction plate with the incision through the periosteum. The method 3800 includes inserting at 3818 a drill sleeve into the central threaded hole of the distraction plate. The method 3800 includes drilling at 3820 a hole into the patient's bone, wherein a drill bit is disposed through the drill sleeve. The method 3800 includes removing at 3822 the drill sleeve and inserting the anchor screw through the central threaded hole of the distraction plate and into the pilot hole drilled into the patient's bone. The method 3800 includes driving at 3824 a preassembled anchor screw and actuation screw through central threaded hole of distraction plate and into pilot hole drilled into patient bone tissue by utilizing cannulated driver until the actuation screw is located flush into the distraction plate, and wherein a magnet is coupled to the actuation screw. The method 3800 includes closing at 3826 patient tissue over the device to reduce a likelihood of infection. The method 3800 includes placing at 3828 an external magnetic controller over the device and establishing a magnetic coupling between the magnet coupled to the actuation screw (installed within the patient) and an external magnet of the external magnetic controller. The method 3800 includes rotating at 3830 the external magnet of the external magnetic controller to cause synchronous rotation of the magnet coupled to the actuation screw.

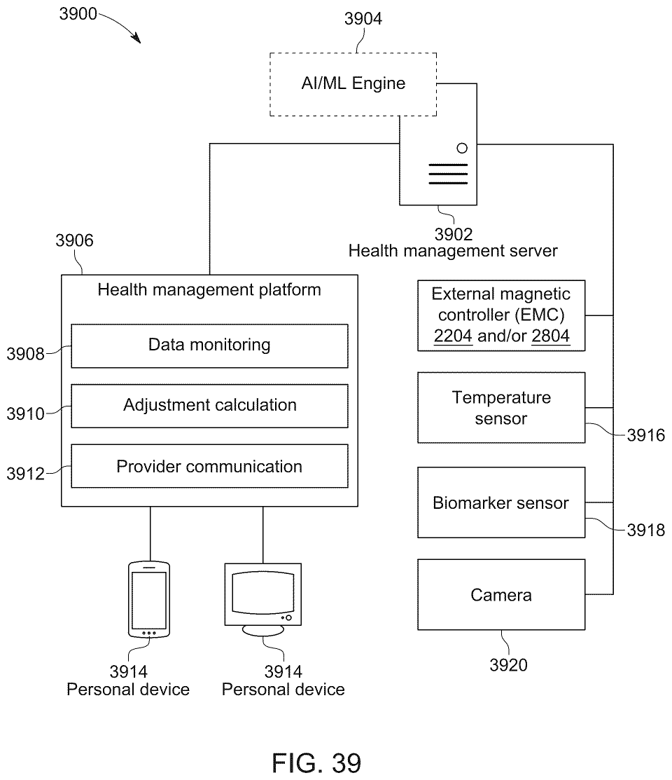

FIG. 39 is a schematic block diagram of a system 3900 for remotely monitoring and controlling a distraction device. The system 3900 may be utilized in connection with any of the systems, methods, or devices described herein. The system 3900 may specifically be utilized in connection with any of the distraction devices described herein.

The system 3900 includes a health management server 3902 that processes operations for a health management platform 3906. The health management server 3902 renders a graphical user interface (GUI) that is made accessible by way of the health management platform 3906. The health management platform 3906 is made accessible to one or more personal devices 3914 by way of a computer-executed application, web browser, or other means. The health management platform 3906 may be accessed with a personal device 3914 that is in communication with a network, such as wide area network, local area network, the Internet, and so forth.

The health management server 3902 may optionally include an artificial intelligence and/or machine learning (AI/ML) engine 3904. The AI/ML engine 3904 is trained upon a set of training data. The AI/ML engine 3904 may be trained to assess data output by any of the EMC 2204, 2804, a temperature sensor 3916, a biomarker sensor 3918, and/or a camera 3920 to monitor the status of a distraction device and determine whether there is a likely issue with the patient. The AI/ML engine 3904 may further be trained on data from numerous orthopedic procedures, including osteotomy and distraction histogenesis procedures, to generate a proposed treatment protocol for adjusting a fixator with an automated fixator adjustor that may be integrated into the EMC 2204, 2804.

The health management platform 3906 may include modules for providing information regarding data monitoring 3908, adjustment calculation 3910, provider communication 3912, and so forth. The health management platform 3906 may be rendered on an application that is run on a personal device 3914, such as a mobile phone, a tablet, or other personal computer. The health management platform 3906 may be rendered on a web browser that is accessible by way of a personal device 3914 connected to the Internet or LoRaWAN.

Patients, healthcare providers, administrators, and other users may access the health management platform 3906 with a personal device 3914 to view up-to-date data provided by any of the temperature sensor 3916, biomarker sensor 3918, or camera 3920. Additionally, the health management platform 3906 provides current and past information regarding adjustment calculations 3910 for a fixator that is currently or was previously fixated to a patient. The adjustment calculations 3910 may be utilized to change or maintain protocols to be implemented by an automated fixator adjustor integrated into the EMC 2204, 2804. The health management platform 3912 provides a means for secure bidirectional communication with patients and healthcare providers by way of the provider communication 3912 module. The health management platform 3912 additionally provides a means for secure bidirectional communication with devices such as the EMC 2204, 2804, temperature sensor 3916, biomarker sensor 3918, and the camera 3920.

The EMC 2204, 2804 may be automated to engage the magnetic and cause distraction or retraction without manual user intervention. The EMC 2204, 2804 may include one or more processors configured to execute instructions stored in non-transitory computer readable storage medium. The processors of the EMC 2204, 2804 may receive instructions from any of the health management server 3902, a locally stored memory device with the EMC 2204, 2804, or from another source such as the personal device 3914. The EMC 2204, 2804 may automatically lift or lower the compliant elongated member of a distraction device to make real-time adjustments in accordance with a healthcare provider's protocol. A healthcare provider may amend the adjustment protocol by communicating with the health management server 3902 by way of the health management platform 3906.

The EMC 2204, 2804 may include one or more Bluetooth® low energy chips and/or near-field communication chips that enable the EMC 2204, 2804 may wirelessly communicate with personal devices 3914 by way of Bluetooth® or near-field communication. The EMC 2204, 2804 may further be equipped with Wi-Fi® or LoRaWAN capability that enables the EMC 2204, 2804 to wirelessly communicate with the health management server 3902 directly.

The EMC 2204, 2804 may be automated to rotate the magnet and cause distraction or retraction of implant without manual user intervention. The EMC 2204, 2804 may include one or more processors configured to execute instructions stored in non-transitory computer readable storage medium. The processors of the EMC 2204, 2804 may receive instructions from any of the health management server 3902, a locally stored memory device with the external magnetic controller 1700, or from another source such as the personal device 3914. The EMC 2204, 2804 may automatically lift or lower the compliant elongated member of a distraction device to make real-time adjustments in accordance with a healthcare provider's protocol. A healthcare provider may amend the adjustment protocol by communicating with the health management server 3902 by way of the health management platform 3906, or through the personal device 3914.

The temperature sensor 3916 may be temporarily installed within a patient to monitor the real-time internal temperature of the patient. Data output by the temperature sensor 3916 may be assessed to determine if patient tissue is inflamed or if the patient may have an infection at the surgical site.

The biomarker sensor 3918 may be temporarily installed within a patient to monitor the real-time presence of certain biomarkers. The biomarker sensor 3918 may specifically be tuned to monitor the presence of biomarkers that may signal the patient currently has an infection at the surgical site. The data output by the biomarker sensor 3918 may be utilized to determine if the patient likely has an infection at the surgical site and requires additional care.

The camera 3920 may include a standalone camera or one or more image sensors, light sources, photocells, other sensors, and other features associated with another computing device such as a mobile phone. In some cases, a personal device 3914 comprising a camera communicates with the health management server 3902 by way of a computer-executed application. The health management server 3902 executes the application and may communicate directly with the camera 3920 to receive images and other data captured by the camera 3920. In some cases, the camera 3920 may integrated with any of the systems described herein and may capture up-to-date pictures of a fixator installed on a patient.

The EMC 2204, 2804 comprises one or more processors configured to execute instructions stored in non-transitory computer readable storage medium. The processors of the EMC 2204, 2804 may receive instructions from any of the health management server 3902, a locally stored memory device with the EMC 2204, 2804, or from another source such as the personal device 3914. A healthcare provider may amend the adjustment protocol by communicating with the health management server 3902 by way of the health management platform 3906.

The EMC 2204, 2804 may include one or more Bluetooth® low energy chips and/or near-field communication chips that enable the EMC 2204, 2804 to wirelessly communicate with personal devices 3914 by way of Bluetooth® or near-field communication. The EMC 2204, 2804 may further be equipped with Wi-Fi® or LoRaWAN capability that enables the EMC 2204, 2804 to wirelessly communicate with the health management server 3902 directly.

The temperature sensor 3916, biomarker sensor 3916, and camera 3920 may include separate components that individually communicate with the health management server 3902 over a network connection. In other implementations, one or more of the temperature sensor 3916, the biomarker sensor 3918, or the camera 3920 may be integrated into the EMC 2204, 2804.

The personal device 3914 is any personal computing device that can communicate with the health management server 3902. The personal device 3914 may include a smart phone, a tablet, a laptop, a personal computer, virtual or augmented reality device, and so forth. The personal devices 3914 may communicate with the health management server 3902 by way of a local area network (LAN) connection, a wide area network (WAN) connection, or another network connection.

The personal device 3914 may include an application installed thereon for enabling streamlined communication with the health management server 3902. The application is configured for multiple platforms, including, for example, smart phone platforms, tablet platforms, Android® operating systems, Linux® operating systems, Windows® operating systems, iOS® operating systems, Macintosh® operating systems, and so forth. The application is configured to provide secure bidirectional communication with one or more of the health management server 3902, the EMC 2204, 2804, the temperature sensor 3916, the biomarker sensor 3918, or the camera 3920. The application may wirelessly receive sensor information, system status information, and may further send commanding signals to sensors and system components. The application is configured to display system information through one or more means, including, for example, graphic displays, audible sounds, videos, and so forth. The application is configured to receive user input and is capable of communicating with multiple systems.

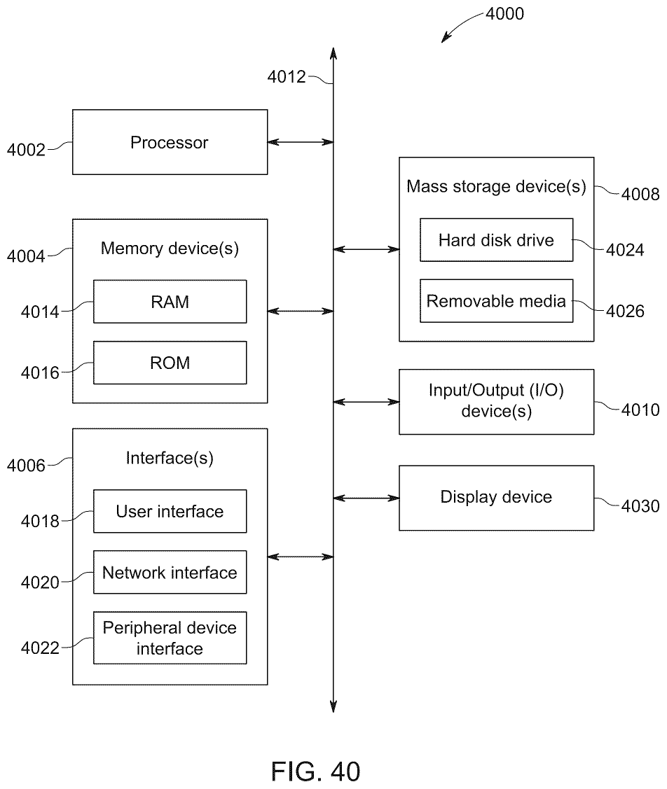

Referring now to FIG. 40, a block diagram of an example computing device 4000 is illustrated. Computing device 4000 may be used to perform various procedures, such as those discussed herein. Computing device 4000 can perform various monitoring functions as discussed herein, and can execute one or more application programs, such as the application programs or functionality described herein. Computing device 4000 can be any of a wide variety of computing devices, such as a desktop computer, in-dash computer, vehicle control system, a notebook computer, a server computer, a handheld computer, tablet computer and the like.

Computing device 4000 includes one or more processor(s) 4012, one or more memory device(s) 4004, one or more interface(s) 4006, one or more mass storage device(s) 4008, one or more Input/output (I/O) device(s) 4010, and a display device 4030 all of which are coupled to a bus 4012. Processor(s) 4012 include one or more processors or controllers that execute instructions stored in memory device(s) 4004 and/or mass storage device(s) 4008. Processor(s) 4012 may also include diverse types of computer-readable media, such as cache memory.

Memory device(s) 4004 include various computer-readable media, such as volatile memory (e.g., random access memory (RAM) 4014) and/or nonvolatile memory (e.g., read-only memory (ROM) 4016). Memory device(s) 4004 may also include rewritable ROM, such as Flash memory.

Mass storage device(s) 4008 include various computer readable media, such as magnetic tapes, magnetic disks, optical disks, solid-state memory (e.g., Flash memory), and so forth. As shown in FIG. 40, a particular mass storage device 4008 is a hard disk drive 4024. Various drives may also be included in mass storage device(s) 4008 to enable reading from and/or writing to the various computer readable media. Mass storage device(s) 4008 include removable media 4026 and/or non-removable media.

I/O device(s) 4010 include various devices that allow data and/or other information to be input to or retrieved from computing device 4000. Example I/O device(s) 4010 include cursor control devices, keyboards, keypads, microphones, monitors, touchscreen devices, or other display devices, speakers, printers, network interface cards, modems, and the like.

Display device 4030 includes any type of device capable of displaying information to one or more users of computing device 4000. Examples of display device 4030 include a monitor, display terminal, video projection device, and the like.

Interface(s) 4006 include various interfaces that allow computing device 4000 to interact with other systems, devices, or computing environments. Example interface(s) 4006 may include any number of different network interfaces 4020, such as interfaces to local area networks (LANs), wide area networks (WANs), wireless networks, and the Internet. Other interface(s) include user interface 4018 and peripheral device interface 4022. The interface(s) 4006 may also include one or more user interface elements 4018. The interface(s) 4006 may also include one or more peripheral interfaces such as interfaces for printers, pointing devices (mice, track pad, or any suitable user interface now known to those of ordinary skill in the field, or later discovered), keyboards, and the like.

Bus 4012 allows processor(s) 4012, memory device(s) 4004, interface(s) 4006, mass storage device(s) 4008, and I/O device(s) 4010 to communicate with one another, as well as other devices or components coupled to bus 4012. Bus 4012 represents one or more of several types of bus structures, such as a system bus, PCI bus, IEEE bus, USB bus, and so forth.

For purposes of illustration, programs and other executable program components are shown herein as discrete blocks, although it is understood that such programs and components may reside at various times in different storage components of computing device 4000 and are executed by processor(s) 4012. Alternatively, the systems and procedures described herein can be implemented in hardware, or a combination of hardware, software, and/or firmware. For example, one or more application specific integrated circuits (ASICs) can be programmed to conduct one or more of the systems and procedures described herein. As used herein, the terms “module” or “component” are intended to convey the implementation apparatus for accomplishing a process, such as by hardware, or a combination of hardware, software, and/or firmware, for the purposes of performing all or parts of operations disclosed herein. The terms “module” or “component” are intended to convey independent in how the modules, components, or their functionality or hardware may be implemented in different embodiments.

### Examples

The following examples pertain to further embodiments.

Example 1 is a system for performing a distraction histogenesis surgical procedure. The system includes an anchor screw comprising a hollow interior defined by a sidewall. The system includes an actuation screw comprising: a magnet comprising a first pole and a second pole; and a screw shaft attached to the magnet. The system is such that the screw shaft is configured to be disposed within the hollow interior defined by the sidewall of the anchor screw.

Example 2 is a system as in Example 1, wherein the anchor screw comprises: external threading on an external surface of the sidewall; and internal threading on an internal surface of the sidewall.

Example 3 is a system as in any of Examples 1-2, wherein the screw shaft of the actuation screw comprises magnet external threading, and wherein the magnet external threading corresponds with the internal threading of the anchor screw.

Example 4 is a system as in any of Examples 1-3, wherein the magnet external threading engages with the internal threading of the anchor screw such that the actuation screw moves up or down along a longitudinal axis of the anchor screw in response to rotation of the magnet.

Example 5 is a system as in any of Examples 1-4, wherein the magnet comprises a bottom surface attached to the screw shaft; wherein the magnet comprises a top surface located opposite to the bottom surface; and wherein the first pole is located adjacent to the second pole such that each of the first pole and the second pole is disposed at the top surface of the magnet.

Example 6 is a system as in any of Examples 1-5, further comprising an external magnetic controller comprising an external magnet configured to magnetically engage with the magnet of the actuation screw, wherein the external magnet comprises: a first external pole configured to magnetically attract to the first pole of the magnet liter; and a second external pole configured to magnetically attract to the second pole of the actuation screw.

Example 7 is a system as in any of Examples 1-6, wherein the external magnet comprises a bottom surface configured to be disposed adjacent to an external tissue of a patient; wherein the first external pole is located adjacent to the second external pole such that each of the first external pole and the second external pole is disposed at the bottom surface of the external magnet.

Example 8 is a system as in any of Examples 1-7, wherein the first pole of the actuation screw is located adjacent to the second pole of the actuation screw such that each of the first pole and the second pole is disposed at a top surface of the magnet; wherein the top surface of the magnet is configured to magnetically engage with the bottom surface of the external magnet in response to magnetic attraction between the first external pole and the first pole, and further in response to magnetic attraction between the second external pole and the second pole.

Example 9 is a system as in any of Examples 1-8, wherein rotation of the external magnet causes rotation of the actuation screw in response to magnetic attraction between: the first external pole of the external magnet and the first pole of the actuation screw; and the second external pole of the external magnet and the second pole of the actuation screw.

Example 10 is a system as in any of Examples 1-9, wherein the first pole of the actuation screw and the first external pole of the external magnetic controller comprise opposite polarity; and wherein the second pole of the actuation screw and the second external pole of the external magnetic controller comprise opposite polarity.

Example 11 is a system as in any of Examples 1-10, wherein rotation of the external magnet of the external magnetic controller causes rotation of the magnet of the actuation screw, in response to establishing an attraction between the external magnet and the magnet.

Example 12 is a system as in any of Examples 1-11, wherein the anchor screw is configured to be screwed into a tissue of a patient and remain stationary within the tissue of the patient; and wherein the screw shaft is configured to rotate within the hollow interior of the anchor screw to cause the actuation screw to move up or down along a longitudinal axis of the anchor screw and relative to a position of the anchor screw.

Example 13 is a system as in any of Examples 1-12, wherein the anchor screw is configured to be screwed into a bone tissue of a patient and remain stationary within the bone tissue of the patient; wherein the magnet of the actuation screw is configured to be disposed adjacent and underneath a periosteum of the patient; and wherein the screw shaft is configured to rotate within the hollow interior of the anchor screw to cause the actuation screw to move up or down along a longitudinal axis of the anchor screw and relative to a position of the anchor screw, to cause distraction or retraction of the periosteum of the patient.

Example 14 is a system as in any of Examples 1-13, wherein the anchor screw is configured to be screwed into a first tissue of a patient; and wherein a position of the actuation screw relative to a longitudinal axis of the anchor screw is adjusted and maintained by rotating the screw shaft within the hollow interior of the anchor screw.

Example 15 is a system as in any of Examples 1-14, wherein the position of the actuation screw is optimized and adjusted to cause distraction or retraction of a second tissue of the patient.

Example 16 is a system as in any of Examples 1-15, further comprising an external magnetic controller configured to cause rotation of the actuation screw, wherein the external magnetic controller comprises: an external magnet configured to magnetically engage with the magnet of the actuation screw; a motor driver; a power source comprising a battery or external power source; a processor; and a motor in mechanical communication with the external magnet and configured to rotate the external magnet; wherein the processor is in electronic communication with the motor and causes the motor to rotate the external magnet according to a schedule via the motor driver.

Example 17 is a system as in any of Examples 1-16, wherein the external magnetic controller further comprises an antenna for receiving instructions over a network, and wherein the instructions comprise the schedule.

Example 18 is a system as in any of Examples 1-17, wherein the external magnetic controller further comprises one or more of a temperature sensor, a biomarker sensor, or a camera.

Example 19 is a system for performing a distraction histogenesis surgical procedure. The system includes an external magnetic controller comprising an external magnet. The system includes an anchor screw comprising a hollow interior defined by a sidewall. The system includes an actuation screw comprising: a magnet comprising a first pole and a second pole; and a screw shaft attached to the magnet. The system is such that the screw shaft is configured to be disposed within the hollow interior defined by the sidewall of the anchor screw. The system is such that the external magnet is configured to magnetically engage with the magnet of the magnetic lifter such that rotation of the external magnet causes rotation of the actuation screw.

Example 20 is a method for performing a distraction histogenesis surgical procedure. The method includes installing an anchor screw in a first tissue of a patient, wherein the anchor screw comprises a hollow interior defined by a sidewall. The method includes installing an actuation screw within the hollow interior of the anchor screw, wherein the actuation screw comprises: a magnet comprising a first pole and a second pole; and a screw shaft attached to the magnet. The method includes causing an external magnet to magnetically engage with the magnet of the actuation screw. The method includes rotating the external magnet to cause rotation of the magnet, and thereby adjust a position of the actuation screw along a longitudinal axis of the anchor screw.

Example 21 is a system. The system includes an anchor screw comprising a hollow interior defined by a sidewall, wherein the anchor screw comprises internal actuation threading attached to an interior surface of the sidewall and further comprises external anchor threading attached to an exterior surface of the sidewall. The system includes an actuation screw comprising a screw shaft that comprises external actuation threading. The system includes a magnet coupled to the actuation screw. The system is such that the external actuation threading of the screw shaft corresponds with the internal actuation threading of the anchor screw. The system is such that rotation of the magnet causes synchronous rotation of the actuation screw.

Example 22 is a system as in Example 21, wherein the rotation of the magnet causes the synchronous rotation of the actuation screw and thereby causes the actuation screw to screw into or out of the hollow interior defined by the sidewall of the anchor screw.

Example 23 is a system as in any of Examples 21-22, further comprising a distraction plate coupled to the actuation screw, wherein the distraction plate is axially coupled to the actuation screw such that: the distraction plate lifts away from the anchor screw in response to the synchronous rotation of the actuation screw causing the actuation screw to screw out of the hollow interior of the anchor screw; the distraction plate lowers toward the anchor screw in response to the synchronous rotation of the actuation screw causing the actuation screw to screw into the hollow interior of the anchor screw.

Example 24 is a system as in any of Examples 21-23, further comprising a coupler that couples the distraction plate to the actuation screw, wherein the coupler permits the actuation screw to rotate independently of the distraction plate such that the distraction plate remains rotationally stationary in response to the synchronous rotation of the actuation screw causing the actuation screw to screw into or out of the hollow interior of the anchor screw.

Example 25 is a system as in any of Examples 21-24, further comprising: a distraction plate comprising a threaded hole that comprises internal threading; and a coupler comprising a coupler sidewall defining a hollow interior, wherein the coupler comprises external threading attached to an exterior surface of the coupler sidewall; wherein the internal threading of the threaded hole of the distraction plate corresponds with the external threading of the coupler.

Example 26 is a system as in any of Examples 21-25, wherein the coupler further comprises a smooth surface on the interior surface of the coupler sidewall.

Example 27 is a system as in any of Examples 21-26, wherein the screw shaft of the actuation screw further comprises a smooth portion that does not comprise the external actuation threading; and wherein the coupler is configured to couple the actuation screw to the distraction plate such that: the coupler is screwed into the threaded hole of the distraction plate by interfacing the external threading of the coupler with the internal threading of the threaded hole; and the actuation screw is disposed within the hollow interior of the coupler such that the smooth portion of the screw shaft of the actuation screw rotates freely within the hollow interior of the coupler.

Example 28 is a system as in any of Examples 21-27, wherein the coupler is configured to couple the actuation screw to the distraction plate such that: the distraction plate is axially coupled to the actuation screw such that the distraction plate lifts away from the anchor screw in response to the synchronous rotation of the actuation screw causing the actuation screw to screw out of the hollow interior of the anchor screw; the distraction plate is axially coupled to the actuation screw such that the distraction plate lowers toward the anchor screw in response to the synchronous rotation of the actuation screw causing the actuation screw to screw into the hollow interior of the anchor screw; and the distraction plate is not rotationally coupled to the actuation screw such that the distraction plate remains rotationally stationary in response to the synchronous rotation of the actuation screw causing the actuation screw to screw out of or into the hollow interior of the anchor screw.

Example 29 is a system as in any of Examples 21-28, wherein the magnet comprises: a circular cross-sectional geometry; a first pole comprising a first polarity; and a second pole comprising a second polarity; wherein the first polarity is opposite to the second polarity; and wherein the circular cross-sectional geometry comprises each of the first pole and the second pole such that each of the first pole and the second pole comprises a half-circle cross-sectional geometry.

Example 30 is a system as in any of Examples 21-29, wherein the magnet comprises: a circular cross-sectional geometry; a plurality of a first pole comprising a first polarity; and a plurality of a second pole comprising a second polarity; wherein the first polarity is opposite to the second polarity; wherein the circular cross-sectional geometry comprises the plurality of the first pole and the plurality of the second pole arranged in an alternating orientation such that each of the plurality of the first pole is adjacent to two of the plurality of second pole, and further such that each of the plurality of the second pole is adjacent to two of the plurality of the first pole; and wherein each of the plurality of the first pole and each of the plurality of the second pole comprises a circular sector cross-sectional geometry.

Example 31 is a system as in any of Examples 21-30, wherein the magnet comprises a plate magnet comprises a top surface and a bottom surface, wherein the bottom surface is coupled to the actuation screw, and wherein each of a first polarity and a second polarity of the magnet is present on the top surface.

Example 32 is a system as in any of Examples 21-31, wherein the magnet comprises an elongated magnetic core disposed within a hollow interior defined by the actuation screw.

Example 33 is a system as in any of Examples 21-32, wherein the magnet is a component of the actuation screw, and wherein the magnet comprises an elongated magnetic core forming a component of the actuation screw.

Example 34 is a system as in any of Examples 21-33, further comprising an external magnetic controller comprising an external magnet configured to magnetically engage with the magnet, wherein rotation of the external magnet comprises synchronous rotation of the magnet, and thereby further causes the synchronous rotation of the actuation screw.

Example 35 is a system as in any of Examples 21-34, wherein the external magnet comprises a bottom surface configured to be disposed adjacent to an external tissue of a patient; wherein a first external pole of the external magnet is located adjacent to a second external pole of the external magnet such that each of the first external pole and the second external pole is disposed at the bottom surface of the external magnet.

Example 36 is a system as in any of Examples 21-35, wherein a first pole of the magnet is located adjacent to a second pole of the magnet such that each of the first pole and the second pole is disposed at a top surface of the magnet; and wherein the top surface of the magnet is configured to magnetically engage with the bottom surface of the external magnet in response to establishing a magnetic attraction between the magnet and the external magnet.

Example 37 is a system as in any of Examples 21-36, wherein the anchor screw is configured to be screwed into a tissue of a patient and remain stationary within the tissue of the patient; and wherein the screw shaft of the actuation screw is configured to rotate within the hollow interior of the anchor screw to cause the actuation screw to move up or down along a longitudinal axis of the anchor screw and relative to a position of the anchor screw.

Example 38 is a system as in any of Examples 21-37, wherein the anchor screw is configured to be screwed into a bone tissue of a patient and remain stationary within the bone tissue of the patient; wherein the magnet is configured to be disposed underneath a periosteum of the patient; wherein the rotation of the magnet causes the synchronous rotation of the actuation screw such that rotation of the magnet in a first direction causes the actuation screw to distract out of the anchor screw and thereby cause distraction of the periosteum of the patient; and wherein the rotation of the magnet causes the synchronous rotation of the actuation screw such that rotation of the magnet in a second direction causes the actuation screw to retract into the anchor screw and thereby cause retraction of the periosteum of the patient.

Example 39 is a system as in any of Examples 21-38, wherein the anchor screw is configured to be screwed into a tissue substrate of a patient; and wherein a position of the actuation screw relative to a longitudinal axis of the anchor screw is adjusted and maintained by rotating the screw shaft within the hollow interior of the anchor screw.

Example 40 is a system as in any of Examples 21-39, further comprising an external magnetic controller configured to form a magnetic coupling with the magnet, wherein the external magnetic controller comprises: an external magnet configured to form the magnetic coupling with the magnet; a processor; and a motor in mechanical communication with the external magnet, wherein the motor causes rotation of the external magnet, which thereby causes synchronous rotation of the magnet in response to establishing the magnetic coupling, and which further thereby causes the synchronous rotation of the actuation screw; wherein the processor is in electronic communication with the motor and causes the motor to rotate the external magnet according to a schedule.

Example 41 is a system as in any of Examples 21-40, further comprising an external magnetic controller configured to form a magnetic coupling with magnet, wherein the magnet is an implant magnet installed within a patient, and wherein the external magnetic controller comprises: one or more electromagnets with coils configured an electromagnet configured to form the magnetic coupling with the implant magnet; one or more power amplifiers or coil drivers; one or more Hall effect sensors or Hall effect array configured to read a magnetic position of the implant magnet; and a processor; wherein the processor causes actuation of the one or more electromagnets to form the magnetic coupling with the implant magnet and cause synchronized rotation of the implant magnet via the power drivers with feedback from the one or more Hall effect sensors or Hall effect array.

Example 42 is a system as in any of Examples 21-41, wherein the external magnetic controller further comprises an antenna for receiving instructions over a network, and wherein the instructions comprise the schedule.

Example 43 is a system as in any of Examples 21-42, wherein the external magnetic controller further comprises one or more of a temperature sensor, a biomarker sensor, or a camera.

Example 44 is a system for performing a distraction histogenesis surgical procedure, the system comprising: an external magnetic controller comprising an external magnet; and a device configured to be implanted within a patient. The device includes an anchor screw comprising a hollow interior defined by a sidewall, wherein the anchor screw comprises internal actuation threading attached to an interior surface of the sidewall and further comprises external anchor threading attached to an exterior surface of the sidewall. The device includes an actuation screw comprising a screw shaft that comprises external actuation threading. The device includes a magnet coupled to the actuation screw. The device is such that the external actuation threading of the screw shaft corresponds with the internal actuation threading of the anchor screw. The system is such that rotation of the external magnet causes synchronous rotation of the magnet in response to forming a magnetic coupling between the external magnet and the magnet. The device is such that the synchronous rotation of the magnet causes synchronous rotation of the actuation screw.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A system for performing a distraction histogenesis surgical procedure, the system comprising:
an anchor screw comprising a hollow interior defined by a sidewall, wherein the anchor screw comprises internal actuation threading attached to an interior surface of the sidewall and further comprises external anchor threading attached to an exterior surface of the sidewall;
an actuation screw comprising a screw shaft that comprises external actuation threading; and
a magnet coupled to the actuation screw;
wherein the external actuation threading of the screw shaft corresponds with the internal actuation threading of the anchor screw; and
wherein rotation of the magnet causes synchronous rotation of the actuation screw.

2. The system of claim 1, wherein the rotation of the magnet causes the synchronous rotation of the actuation screw and thereby causes the actuation screw to screw into or out of the hollow interior defined by the sidewall of the anchor screw.

3. The system of claim 2, further comprising a distraction plate coupled to the actuation screw, wherein the distraction plate is axially coupled to the actuation screw such that:
the distraction plate lifts away from the anchor screw in response to the synchronous rotation of the actuation screw causing the actuation screw to screw out of the hollow interior of the anchor screw;
the distraction plate lowers toward the anchor screw in response to the synchronous rotation of the actuation screw causing the actuation screw to screw into the hollow interior of the anchor screw.

4. The system of claim 3, further comprising a coupler that couples the distraction plate to the actuation screw, wherein the coupler permits the actuation screw to rotate independently of the distraction plate such that the distraction plate remains rotationally stationary in response to the synchronous rotation of the actuation screw causing the actuation screw to screw into or out of the hollow interior of the anchor screw.

5. The system of claim 1, further comprising:
a distraction plate comprising a threaded hole that comprises internal threading; and
a coupler comprising a coupler sidewall defining a hollow interior, wherein the coupler comprises external threading attached to an exterior surface of the coupler sidewall;
wherein the internal threading of the threaded hole of the distraction plate corresponds with the external threading of the coupler.

6. The system of claim 5, wherein the coupler further comprises a smooth surface on the interior surface of the coupler sidewall.

7. The system of claim 6, wherein the screw shaft of the actuation screw further comprises a smooth portion that does not comprise the external actuation threading; and
wherein the coupler is configured to couple the actuation screw to the distraction plate such that:
the coupler is screwed into the threaded hole of the distraction plate by interfacing the external threading of the coupler with the internal threading of the threaded hole of the distraction plate; and
the actuation screw is disposed within the hollow interior of the coupler such that the smooth portion of the screw shaft of the actuation screw rotates freely within the hollow interior of the coupler.

8. The system of claim 7, wherein the coupler is configured to couple the actuation screw to the distraction plate such that:
the distraction plate is axially coupled to the actuation screw such that the distraction plate lifts away from the anchor screw in response to the synchronous rotation of the actuation screw causing the actuation screw to screw out of the hollow interior of the anchor screw;
the distraction plate is axially coupled to the actuation screw such that the distraction plate lowers toward the anchor screw in response to the synchronous rotation of the actuation screw causing the actuation screw to screw into the hollow interior of the anchor screw; and
the distraction plate is not rotationally coupled to the actuation screw such that the distraction plate remains rotationally stationary in response to the synchronous rotation of the actuation screw causing the actuation screw to screw out of or into the hollow interior of the anchor screw.

9. The system of claim 1, wherein the magnet comprises:
a circular cross-sectional geometry;
a first pole comprising a first polarity; and
a second pole comprising a second polarity;
wherein the first polarity is opposite to the second polarity; and
wherein the circular cross-sectional geometry comprises each of the first pole and the second pole such that each of the first pole and the second pole comprises a half-circle cross-sectional geometry.

10. The system of claim 1, wherein the magnet comprises:
a circular cross-sectional geometry;
a plurality of a first pole comprising a first polarity; and
a plurality of a second pole comprising a second polarity;
wherein the first polarity is opposite to the second polarity;
wherein the circular cross-sectional geometry comprises the plurality of the first pole and the plurality of the second pole arranged in an alternating orientation such that each of the plurality of the first pole is adjacent to two of the plurality of second pole, and further such that each of the plurality of the second pole is adjacent to two of the plurality of the first pole; and
wherein each of the plurality of the first pole and each of the plurality of the second pole comprises a circular sector cross-sectional geometry.

11. The system of claim 1, wherein the magnet comprises a plate magnet comprises a top surface and a bottom surface, wherein the bottom surface is coupled to the actuation screw, and wherein each of a first polarity and a second polarity of the magnet is present on the top surface.

12. The system of claim 1, wherein the magnet comprises an elongated magnetic core disposed within a hollow interior defined by the actuation screw.

13. The system of claim 1, wherein the magnet is a component of the actuation screw, and wherein the magnet comprises an elongated magnetic core forming a component of the actuation screw.

14. The system of claim 1, further comprising an external magnetic controller comprising an external magnet configured to magnetically engage with the magnet, wherein rotation of the external magnet comprises synchronous rotation of the magnet, and thereby further causes the synchronous rotation of the actuation screw.

15. The system of claim 14, wherein the external magnet comprises a bottom surface configured to be disposed adjacent to an external tissue of a patient;
wherein a first external pole of the external magnet is located adjacent to a second external pole of the external magnet such that each of the first external pole and the second external pole is disposed at the bottom surface of the external magnet.

16. The system of claim 15, wherein a first pole of the magnet is located adjacent to a second pole of the magnet such that each of the first pole and the second pole is disposed at a top surface of the magnet; and
wherein the top surface of the magnet is configured to magnetically engage with the bottom surface of the external magnet in response to establishing a magnetic attraction between the magnet and the external magnet.

17. The system of claim 1, wherein the anchor screw is configured to be screwed into a tissue of a patient and remain stationary within the tissue of the patient; and
wherein the screw shaft of the actuation screw is configured to rotate within the hollow interior of the anchor screw to cause the actuation screw to move up or down along a longitudinal axis of the anchor screw and relative to a position of the anchor screw.

18. The system of claim 1, wherein the anchor screw is configured to be screwed into a bone tissue of a patient and remain stationary within the bone tissue of the patient;
wherein the magnet is configured to be disposed underneath a periosteum of the patient;
wherein the rotation of the magnet causes the synchronous rotation of the actuation screw such that rotation of the magnet in a first direction causes the actuation screw to distract out of the anchor screw and thereby cause distraction of the periosteum of the patient; and
wherein the rotation of the magnet causes the synchronous rotation of the actuation screw such that rotation of the magnet in a second direction causes the actuation screw to retract into the anchor screw and thereby cause retraction of the periosteum of the patient.

19. The system of claim 1, wherein the anchor screw is configured to be screwed into a tissue substrate of a patient; and
wherein a position of the actuation screw relative to a longitudinal axis of the anchor screw is adjusted and maintained by rotating the screw shaft within the hollow interior of the anchor screw.

20. The system of claim 1, further comprising an external magnetic controller configured to form a magnetic coupling with the magnet, wherein the external magnetic controller comprises:
an external magnet configured to form the magnetic coupling with the magnet;
a processor; and
a motor driver in mechanical communication with the external magnet, wherein the motor causes rotation of the external magnet, which thereby causes synchronous rotation of the magnet in response to establishing the magnetic coupling, and which further thereby causes the synchronous rotation of the actuation screw;
wherein the processor is in electronic communication with the motor and causes the motor to rotate the external magnet according to a schedule and via the motor driver.

21. The system of claim 1, further comprising an external magnetic controller configured to form a magnetic coupling with the magnet, wherein the external magnetic controller comprises:
one or more electromagnets configured to form the magnetic coupling with the magnet;
a Hall effect sensor configured to read a magnetic position of the magnet;
one or more power drivers; and
a processor;
wherein the processor controls synchronized actuation of the one or more electromagnets to form the magnetic coupling with the magnet and cause synchronized rotation of the magnet via the one or more power drivers with feedback from the Hall effect sensor.

22. The system of claim 20, wherein the external magnetic controller further comprises an antenna for receiving instructions over a network, and wherein the instructions comprise the schedule.

23. The system of claim 20, wherein the external magnetic controller further comprises one or more of a temperature sensor, a biomarker sensor, or a camera.

24. A system for performing a distraction histogenesis surgical procedure, the system comprising:
an external magnetic controller comprising an external magnet; and
a device configured to be implanted within a patient, wherein the device comprises:
an anchor screw comprising a hollow interior defined by a sidewall, wherein the anchor screw comprises internal actuation threading attached to an interior surface of the sidewall and further comprises external anchor threading attached to an exterior surface of the sidewall;
an actuation screw comprising a screw shaft that comprises external actuation threading; and
a magnet coupled to the actuation screw;
wherein the external actuation threading of the screw shaft corresponds with the internal actuation threading of the anchor screw;
wherein rotation of the external magnet causes synchronous rotation of the magnet in response to forming a magnetic coupling between the external magnet and the magnet; and
wherein rotation of the magnet causes synchronous rotation of the actuation screw.
